(19) United States
(12) Patent Application Publication
(10) Pub. No.: US 2003/0087820 A1
(43) Pub. Date: May 8, 2003

(54) Novel exendin agonist formulations and methods of administration thereof

(76) Inventors: Andrew A. Young, La Jolla, CA (US); Orville G. Kolterman, Poway, CA (US)

Correspondence Address: Molly A. Holman, Ph.D., Amylin Pharmaceuticals, Inc., 9373 Towne Centre Drive, San Diego, CA, 92121, US

(21) Appl. No.: 10/157,224

(22) Filed: May 28, 2002

Related U.S. Application Data
(63) Continuation-in-part of application No. 09/889,330, filed on Dec. 27, 2001.
Application No. 09/889,330 is a 371 of international application No. PCT/US00/00902, filed on Jan. 14, 2000.
(60) Provisional application No. 60/116,380, filed on Jan. 14, 1999.
(60) Provisional application No. 60/175,365, filed on Jan. 10, 2000.

(51) Int. Cl. A61K 38/17

(52) U.S. Cl. 514/12

(57) ABSTRACT

Novel exendin and exendin agonist compound formulations and dosages and methods of administration thereof are provided. These compositions and methods are useful in treating diabetes and conditions that would be benefited by lowering plasma glucose or delaying and/or slowing gastric emptying or inhibiting food intake.

### CROSS-REFERENCE TO RELATED APPLICATIONS

This application is a continuation-in-part of Ser. No. 09/889,330, entitled “Novel Exendin Agonist Formulations and Methods of Administration Thereof,” filed Jul. 13, 2001, which claims priority from PCT/US00/00902, also entitled “Novel Exendin Agonist Formulations and Methods of Administration Thereof,” filed Jan. 14, 2000, which claims priority from U.S. Provisional Application No. 60/116,380, entitled “Novel Exendin Agonist Formulations And Methods of Administration Thereof,” filed Jan. 14, 1999, and U.S. Provisional Application No. 60/175,365, entitled “Use of Exendins and Agonists Thereof for Modulation of Triglyceride Levels and Treatment of Dyslipidemia,” filed Jan. 10, 2000, the contents of all of which are hereby incorporated by reference in their entireties.

### FIELD OF THE INVENTION

The present invention relates to novel exendin and peptide exendin agonist formulations, dosages, and dosage formulations that are bioactive and are deliverable by any means.

### BACKGROUND

The following description includes information that may be useful in understanding the present invention. It is not an admission that any of the information provided herein is prior art to the presently claimed inventions, or relevant, nor that any of the publications specifically or implicitly referenced are prior art.

The exendins are peptides that are found in the salivary secretions of the Gila monster and the Mexican Beaded Lizard. Exendin-3 [SEQ. ID. NO. 1: His Ser Asp Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala Pro Pro Pro Ser-NH₂] is present in the salivary secretions of Heloderma horridum (Mexican Beaded Lizard), and exendin-4 [SEQ. ID. NO. 2: His Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala Pro Pro Pro Ser-NH₂] is present in the salivary secretions of Heloderma suspectum (Gila monster)(Eng, J., et al., J. Biol. Chem., 265:20259-62, 1990; Eng, J., et al., J. Biol. Chem., 267:7402-05, 1992).

Exendin-4 reportedly can stimulate somatostatin release and inhibit gastrin release in isolated stomachs (Goke, et al., J. Biol. Chem. 268:19650-55, 1993; Schepp, et al., Eur. J. Pharmacol., 69:183-91, 1994; Eissele, et al., Life Sci., 55:629-34, 1994). Exendin-3 and exendin-4 were reportedly found to stimulate cAMP production in, and amylase release from, pancreatic acinar cells (Malhotra, R., et al., Regulatory Peptides, 41:149-56, 1992; Raufman, et al., J. Biol. Chem. 267:21432-37, 1992; Singh, et al., Regul. Pept. 53:47-59, 1994).

Based on their insulinotropic activities, the use of exendin-3 and exendin-4 for the treatment of diabetes mellitus and the prevention of hyperglycemia has been proposed (Eng, U.S. Pat. No. 5,424,286).

Exendin-4 also has a significantly longer duration of action than GLP-1, a mammalian peptide that exhibits some similar glucose-lowering effects as exendin-4. Exendins are not homologous to mammalian GLP-1 (Chen and Drucker, J. Biol. Chem. 272(7):4108-15 (1997)). The observation that the Gila monster also has separate genes for proglucagon (from which GLP-1 is processed) that are more similar to mammalian proglucagon than exendin indicates that exendins are not species homologs of GLP-1.

Various uses for exendin and exendin agonists, such as for regulating gastrointestinal motility (PCT/US97/14199), reducing food intake (PCT/US98/00449) and inotropic and diuretic effects (PCT/US99/02554) have been suggested. Novel exendin agonist compounds have been described in e.g., PCT/US98/16387, PCT/US98/24210, and PCT/US98/24273.

Delivery of peptide drugs is often difficult because of factors such as molecular size, susceptibility to proteolytic breakdown, rapid plasma clearance, peculiar dose-response curves, immunogenicity, bioincompatibility, and the tendency of peptides and proteins to undergo aggregation, adsorption, and denaturation. Thus, there continues to exist a need for the development of alternative methods to the inconvenient, sometimes painful, injection for administration of peptide drugs, such as exendins and the peptide exendin agonist analogs referenced above.

### SUMMARY OF THE INVENTION

In addition to formulations and dosages useful in the administration of exendins and exendin agonists by injection, formulations, dosage formulations, and methods that solve these problems and that are useful in the non-injection delivery of therapeutically effective amounts of exendin and exendin agonists are described and claimed herein.

It has been discovered that even lower plasma levels of exendin and exendin agonists than previously known or suspected are effective to reduce blood glucose, particularly when continuously administered over at least one hour, more preferably at least 2-24 hours, most preferably from 1 day to 4 months. In order to achieve the most preferable administration, formulations and methods are required that will provide a continuous release or delivery of exendin and exendin agonists for the administration period of interest. Examples of these include, an infusion pump, continuous infusion, controlled release formulations utilizing polymer, oil or water insoluble matrices.

Surprisingly low doses and plasma levels of exendins and agonists have been found to produce therapeutic results. Methods of administration of exendins and agonists to patients in need thereof are provided. Such patients include those who have diabetes mellitus, have impaired glucose tolerance, are obese, hyperglycemic, or have dyslipidemia and/or cardiovascular disease. Doses from about 0.0005 μg/kg/dose to about 12000 μg/kg/dose, depending on mode of administration, can be used to achieve therapeutic plasma levels (at least 5 pg/ml, preferably at least 40 pg/ml). Preferably, peak plasma levels do not exceed about 500 pg/ml, more preferably about 250 pg/ml, and most preferably about 150 pg/ml.

Administered parenterally, exendins and agonists in an amount from about 0.001 μg/kg/dose to about 1.0 μg/kg/dose produce therapeutic effects. Bolus or chronic subcutaneous administration is preferred, for example by infusion or slow release matrix. Slow release is that occurring over at least one hour, preferably at least one day, one week, or one month, with longer periods of release being contemplated. Ideally, release is uniform, but variations in the release profile are acceptable. If not administered continuously, preferably exendins and agonists are administered from one to four times per day, preferably two times per day.

If not by a parenteral route of administration, exendin or agonist can be administered via a nasal, oral, buccal, sublingual, intra-tracheal, trans-dermal, trans-mucosal, pulmonary or any other route known in the art.

The invention features pharmaceutical compositions comprising exendins or exendin agonists, particularly peptides (but not limited to peptides) in an extended release formulation, which is capable of releasing the peptide over a predetermined release period of at least one hour ) in an amount such that plasma levels in humans of at least 5 pg/ml are achieved for at least 25% of the predetermined release period, more preferably 50%, 75%, or 90% of the release period. Preferably, average sustained plasma levels (meaning the average of at least two plasma levels taken within the predetermined release period, for example at the beginning, end, or intermediate times) are at least 40 pg/ml over 25-100% of the predetermined release period.

By an “exendin agonist” is meant a compound that mimics one or more effects of exendin, for example, by binding to a receptor where exendin causes one or more of these effects, or by activating a signaling cascade by which exendin causes one or more of these effects. Exendin agonists include exendin agonist peptides, such as analogs and derivatives of exendin-3 and exendin-4 that have one or more desired activities of exendin. Various exendin agonist analogs are identified or referenced herein. Molecules for use in the formulations of the invention include, however, peptides and peptide fragments derived from any source, and small molecules, which act as exendin agonists or antagonists.

According to another aspect, the present invention provides novel exendin agonist compound formulations and dosages, and methods for the administration thereof, that are useful in treating diabetes (including type 1 and type 2 diabetes), obesity, and other conditions that will benefit from the administration of a therapy that can slow gastric emptying, lower plasma glucose levels, and reduce food intake.

The invention also includes methods for treatment of subjects in order to increase insulin sensitivity by administering an exendin or an exendin agonist. The exendin and exendin agonist formulations and dosages described herein may be used to increase the sensitivity of a subject to endogenous or exogenous insulin.

Other features and advantages of the invention will be apparent from the following description of the preferred embodiments thereof, and from the claims.

In accordance with the present invention and as used herein, the following terms are defined to have the following meanings, unless explicitly stated otherwise. “Pharmaceutically acceptable salt” includes salts of the compounds of the present invention derived from the combination of such compounds and an organic or inorganic acid. In practice the use of the salt form is substantially equivalent to use of the base form. The compounds of the present invention are useful in both free base and salt form, with both forms being considered within the scope of the present invention.

### BRIEF DESCRIPTION OF THE DRAWINGS

FIG. 1 shows that continuously infused exendin at all doses tested lowered mean plasma glucose concentrations compared to placebo.

FIG. 2 depicts the effect of a bolus dose of exendin on plasma glucose in the fasting state.

FIG. 3 shows the effect of a bolus dose of exendin on serum insulin in the fasting state.

FIG. 4 depicts the plasma levels of exendin-4 in rats after intra-tracheal administration.

FIG. 5a depicts the plasma exendin-4 concentration after intra-tracheal instillation in db/db mice.

FIG. 5b depicts the effect of intra-tracheal administration of exendin-4 on plasma glucose in db/db mice.

FIGS. 6a and 6b depict the effect of intra-tracheal administration of exendin-4 on plasma glucose in ob/ob mice.

FIG. 7a depicts the plasma exendin-4 concentration after intra-tracheal instillation into rats.

FIG. 7b depicts the bioavailability of exendin-4 following intra-tracheal instillation into rats.

FIG. 8 depicts plasma exendin-4 concentrations in rats exposed to aerosolized exendin-4. Open box indicates duration of exposure to nebulized exendin.

FIG. 9a depicts the effect of ten minutes of exposure to aerosolized exendin-4 on plasma glucose in db/db mice.

FIG. 9b depicts the plasma exendin-4 concentration after ten minutes of exposure of db/db mice to aerosolized exendin-4.

FIG. 10 depicts plasma exendin-4 concentrations in rats after intra-nasal administration of exendin-4.

FIG. 11 depicts the effect of intra-gastric administration of exendin-4 on plasma glucose in db/db mice.

FIG. 12a depicts the plasma exendin-4 concentration after sublingual administration to db/db mice.

FIG. 12b depicts the effect of sublingual administration of exendin-4 on plasma glucose -5 in db/db mice.

FIG. 12c depicts the plasma exendin-4 concentration after sublingual administration to rats.

FIG. 12d depicts the bioavailability of exendin-4 after sublingual administration.

FIG. 12e depicts the Cmax of sublingual exendin-4.

FIG. 13 depicts the effect of exendin-4 (administered i.p. twice daily) on food intake (a), change in body weight (b) (initial body weight 441±39 g), or change in hemoglobin Alc (c) (7.68±0.20% at 0 weeks). Dose-responses (right panels) are for the means over the last 2 of 6 weeks treatment.

FIG. 14 depicts the plasma glucose concentration (a), glucose infusion rate required to maintain euglycemia (b) and plasma lactate concentration (c) in clamp procedures performed on ZDF rats previously treated for 6 weeks with the specified doses of exendin-4 (i.p. twice daily). Dose-responses for glucose infusion rate and plasma lactate concentration are based upon mean values obtained between 60 and 180 min of the clamp procedure.

FIG. 15 depicts the amino acid sequences for certain exendin agonist compounds useful in the present invention [SEQ ID NOS 9-39].

FIGS. 16 and 17 depict glucose-lowering results from the clinical study described in Example 10.

### DETAILED DESCRIPTION OF THE INVENTION

Exendins and Exendin Agonists

Exendin-3 and Exendin-4 are naturally occurring peptides. Animal testing of exendin-4 has shown that its ability to lower blood glucose persists for several hours. Exendin-4, a 39-amino acid polypeptide, has been synthesized using solid phase synthesis as described herein, 19 and this synthetic material has been shown to be identical to that of native exendin-4. Isolated naturally occurring exendins or recombinantly produced exendins are also completely functional in the methods or compositions of the invention, as is any exendin agonist or analog. Also contemplated is the use of exendin antagonists and antagonist analogs for uses where antagonism of exendin activity is desired.

Various aspects of the nonclinical pharmacology of exendin-4 have been studied. In the brain, exendin-4 binds principally to the area postrema and nucleus tractus solitarius region in the hindbrain and to the subfomical organ in the forebrain. Exendin-4 binding has been observed - in the rat and mouse brain and kidney. The structures to which exendin-4 binds in the kidney are unknown.

A number of other experiments have compared the biologic actions of exendin-4 and GLP-1 and demonstrated a more favorable spectrum of properties for exendin-4. A single subcutaneous dose of exendin-4 lowered plasma glucose in db/db (diabetic) and ob/ob (diabetic obese) mice by up to 40%. In Diabetic Fatty Zucker (ZDF) rats, 5 weeks of treatment with exendin-4 lowered HbA_{1c} (a measure of glycosylated hemoglobin used to evaluate plasma glucose levels) by up to 41%. Insulin sensitivity was also improved by 76% following 5 weeks of treatment in obese ZDF rats. In glucose intolerant primates, dose-dependent decreases in plasma glucose were also observed. See also Example 5, which describes the results of an experiment indicating that exendin is more potent and/or effective than GLP-1 in amplifying glucose-stimulated insulin release. Example 6, furthermore, describes work showing that the ability of exendin-4 to lower glucose in vivo was 3430 times more potent than that of GLP-1.

An insulinotropic action of exendin-4 has also been observed in rodents, improving insulin response to glucose by over 100% in non-fasted Harlan Sprague Dawley (HSD) rats, and by up to ˜10-fold in non-fasted db/db mice. Higher pretreatment plasma glucose concentrations were associated with greater glucose-lowering effects. Thus the observed glucose lowering effect of exendin-4 appears to be glucose-dependent, and minimal if animals are already euglycemic. Exendin-4 treatment is also associated with improvement in glycemic indices and in insulin sensitivity, as described in Examples 7 and 11.

Exendin-4 dose dependently slowed gastric emptying in HSD rats and was 90-fold more potent than GLP-1 for this action. Exendin-4 has also been shown to reduce food intake in NIH/Sw (Swiss) mice following peripheral administration, and was at least 1000 times more potent than GLP-1 for this action. Exendin-4 reduced plasma glucagon concentrations by approximately 40% in anesthetized ZDF rats during hyperinsulinemic, hyperglycemic clamp conditions, but did not affect plasma glucagon concentrations during euglycemic conditions in normal rats. See Example 3. Exendin-4 has been shown to dose-dependently reduce body weight in obese ZDF rats, while in lean ZDF rats, the observed decrease in body weight appears to be transient.

Through effects on augmenting and restoring insulin secretion, as well as inhibiting glucagon secretion, exendin-4 is useful in people with type 2 diabetes who retain the ability to secrete insulin. Its effects on food intake, gastric emptying, other mechanisms that modulate nutrient absorption, and glucagon secretion also support the utility of exendin-4 in the treatment of, for example, obesity, type 1 diabetes, and people with type 2 diabetes who have reduced insulin secretion.

The toxicology of exendin-4 has been investigated in single-dose studies in mice, rats, and monkeys, repeated-dose (up to 28 consecutive daily doses) studies in rats and monkeys and in vitro tests for mutagenicity and chromosomal alterations. To date, no deaths have occurred, and there have been no observed treatment-related changes in hematology, clinical chemistry, or gross or microscopic tissue changes. Exendin-4 was demonstrated to be non-mutagenic, and did not cause chromosomal aberrations at the concentrations tested (up to 5000 μg/mL).

In support of the investigation of the nonclinical pharmacokinetics and metabolism of exendin-4, a number of immunoassays have been developed. A radioimmunoassay with limited sensitivity (˜100 pM) was used in initial pharmacokinetic studies. A two-site IRMA assay for exendin-4 was subsequently validated with a lower limit of quantitation of 15 pM (63 pg/ml), and a validated sandwich-type immunoenzymatic assay (IEMA) assay using mouse monoclonal antibodies had a lower limit of quantitation of 2.5 pg/ml (see Example 1). The bioavailability of exendin-4, given subcutaneously, was found to be approximately 50-80% using the it radioimmunoassay. This was similar to that seen following intraperitoneal administration (48-60%). Peak plasma concentrations (Cₘₐₓ) occurred between 30 and 43 minutes (Tₘₐₓ). Both Cₘₐₓ and AUC values were monotonically related to dose. The apparent terminal half-life for exendin-4 given subcutaneously was approximately 90-110 minutes. This was significantly longer than the 14-41 minutes seen following intravenous dosing. Similar results were obtained using the IRMA assay. Degradation studies with exendin-4 compared to GLP-1 indicate that exendin-4 is relatively resistant to degradation.

Investigation of the structure activity relationship (SAR) to evaluate structures that may relate to the activities of exendin, for its stability to metabolism, and for improvement of its physical characteristics, especially as it pertains to peptide stability and to amenability to alternative delivery systems, has led to the discovery of exendin agonist peptide compounds. Exendin agonists include exendin peptide analogs in which one or more naturally occurring amino acids are eliminated or replaced with another amino acid(s). Preferred exendin agonists are agonist analogs of exendin-4. Particularly preferred exendin agonists include exendin-3 [SEQ ID NO 1], exendin-4 [SEQ ID NO 2], exendin-4 (1-30) [SEQ ID NO 6: His Gly Glu Gly Thr Phe Thr Ser Asp Leu Ser Lys Gln Met Glu Glu Glu Ala Val Arg Leu Phe Ile Glu Trp Leu Lys Asn Gly Gly], exendin-4 (1-30) amide [SEQ ID NO 7: His Gly Glu Gly Thr Phe Thr Ser Asp Leu Ser Lys Gln Met Glu Glu Glu Ala Val Arg Leu Phe Ile Glu Trp Leu Lys Asn Gly Gly-NH₂], exendin-4 (1-28) amide [SEQ ID NO 40: His Gly Glu Gly Thr Phe Thr Ser Asp Leu Ser Lys Gln Met Glu Glu Glu Ala Val Arg Leu Phe Ile Glu Trp Leu Lys Asn-NH₂], Leu, Phe exendin-4 [SEQ ID NO 9: His Gly Glu Gly Thr Phe Thr Ser Asp Leu Ser Lys Gln Leu Glu Glu Glu Ala Val Arg Leu Phe Ile Glu Phe Leu Lys Asn Gly Gly Pro Ser Ser Gly Ala Pro Pro Pro Ser-NH₂], ¹⁴Leu, 21Phe exendin-4 (1-28) amide [SEQ ID NO 41: His Gly Glu Gly Thr Phe Thr Ser Asp Leu Ser Lys Gln Leu Glu Glu Glu Ala Val Arg Leu Phe Ile Glu Phe Leu Lys Asn-NH₂], and ¹⁴Leu,22Ala,21Phe exendin-4 (1-28) amide [SEQ ID NO 8: His Gly Glu Gly Thr Phe Thr Ser Asp Leu Ser Lys Gln Leu Glu Glu Glu Ala Val Arg Leu Ala Ile Glu Phe Leu Lys Asn-NH₂], and those described in International Application No. PCT/US98/16387, filed Aug. 6, 1998, entitled, “Novel Exendin Agonist Compounds,” including compounds of the formula (I) [SEQ ID NO. 3]:

Xaa₁ Xaa₂ Xaa₃ Gly Thr Xaa₄ Xaa₅ Xaa₆ Xaa₇ Xaa₈

Ser Lys Gln Xaa₉ Glu Glu Glu Ala Val Arg Leu

Xaa₁₀ Xaa₁₁ Xaa₁₂ Xaa₁₃ Leu Lys Asn Gly Gly Xaa₁₄

Ser Ser Gly Ala Xaa₁₅ Xaa₁₆ Xaa₁₇ Xaa₁₈-Z

wherein Xaa₁ is His, Arg or Tyr; Xaa₂ is Ser, Gly, Ala or Thr; Xaa₃ is Asp or Glu; Xaa₄ is Phe, Tyr or naphthylalanine; Xaa₅ is Thr or Ser; Xaa₆ is Ser or Thr; Xaa₇ is Asp or Glu; Xaa₈ is Leu, Ile, Val, pentylglycine or Met; Xaa₉ is Leu, Ile, pentylglycine, Val or Met; Xaa₁₀ is Phe, Tyr or naphthylalanine; Xaa₁₁ is Ile, Val, Leu, pentylglycine, tert-butylglycine or Met; Xaa₁₂ is Glu or Asp; Xaa₁₃ is Trp, Phe, Tyr, or naphthylalanine; Xaa₁₄, Xaa₁₅, Xaa₁₆ and Xaa₁₇ are independently Pro, homoproline, 3Hyp, 4Hyp, thioproline, N-alkylglycine, N-alkylpentylglycine or N-alkylalanine; Xaa₁₈ is Ser, Thr or Tyr; and Z is —OH or —NH₂; with the proviso that the compound is not exendin-3 or exendin-4.

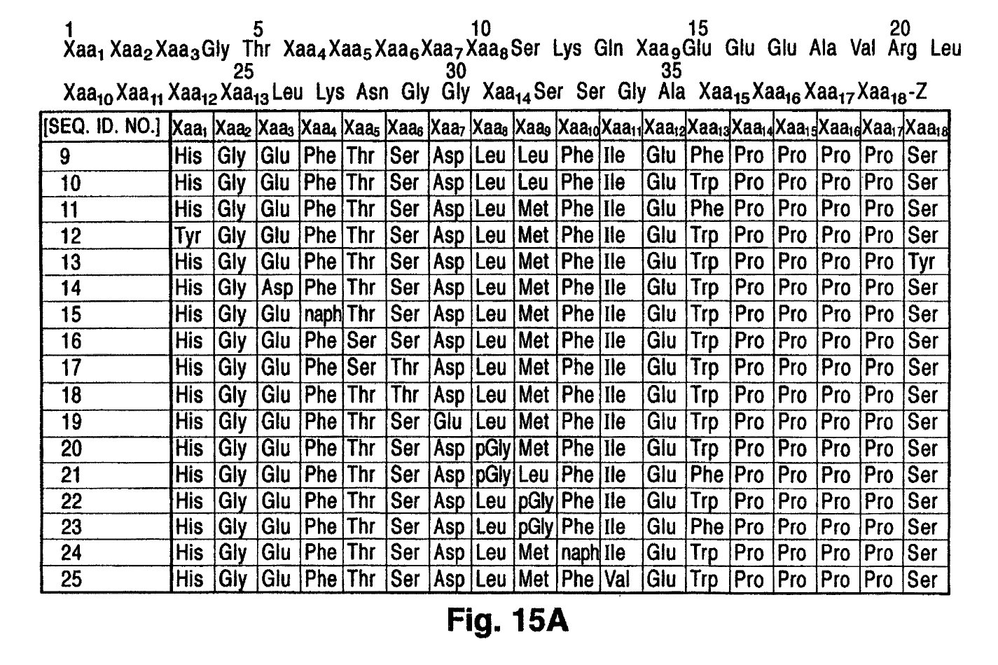

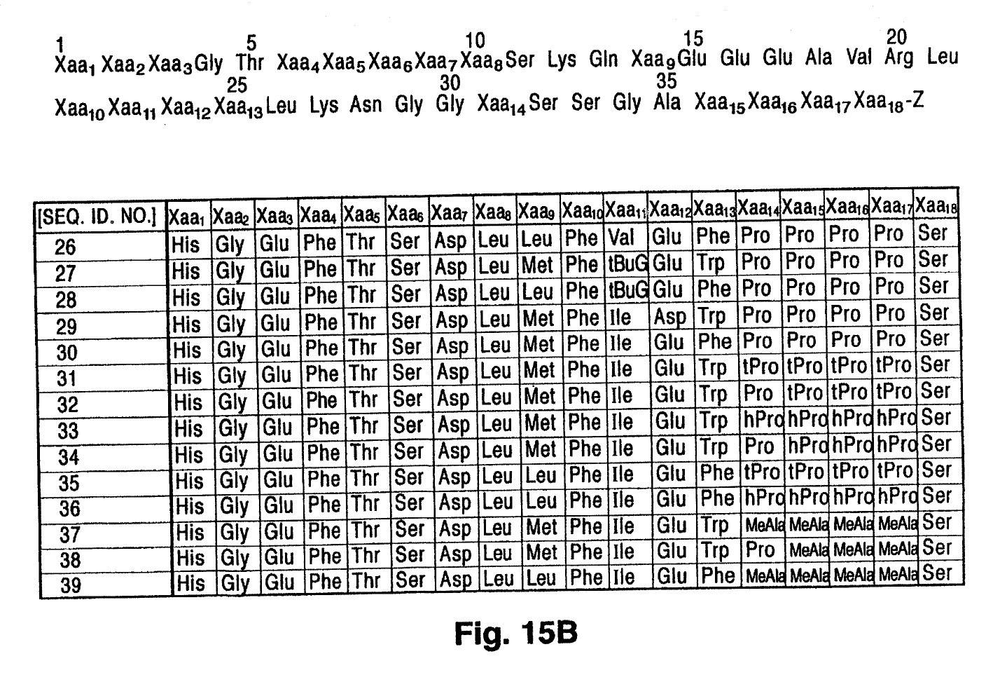

Preferred N-alkyl groups for N-alkylglycine, N-alkylpentylglycine and N-alkylalanine include lower alkyl groups preferably of 1 to about 6 carbon atoms, more preferably of 1 to 4 carbon atoms. Suitable compounds include those listed in FIG. 15 having amino acid sequences of SEQ. ID. NOS. 9 to 39.

Preferred exendin agonist compounds include those wherein Xaa₁ is His or Tyr. More preferably, Xaa₁ is His.

Preferred are those compounds wherein Xaa₂ is Gly.

Preferred are those compounds wherein Xaa₉ is Leu, pentylglycine, or Met.

Preferred compounds include those wherein Xaa₁₃ is Trp or Phe.

Also preferred are compounds where Xaa₄ is Phe or naphthylalanine; Xaa₁₁ is Ile or Val and Xaa₁₄, Xaa₁₅, Xaa₁₆ and Xaa₁₇ are independently selected from Pro, homoproline, thioproline or N-alkylalanine. Preferably N-alkylalanine has a N-alkyl group of 1 to about 6 carbon atoms.

According to an especially preferred aspect, Xaa₁₅, Xaa₁₆ and Xaa₁₇ are the same amino acid reside.

Preferred are compounds wherein Xaa₁₈ is Ser or Tyr, more preferably Ser.

Preferably Z is —NH₂.

According to one aspect, preferred are compounds of formula (I) wherein Xaa₁ is His or Tyr, more preferably His; Xaa₂ is Gly; Xaa₄ is Phe or naphthylalanine; Xaa₉ is Leu, pentylglycine or Met; Xaa₁₀ is Phe or naphthylalanine; Xaa₁₁ is Ile or Val; Xaa₁₄, Xaa₁₅, Xaa₁₆ and Xaa₁₇ are independently selected from Pro, homoproline, thioproline or N-alkylalanine; and Xaa₁₈ is Ser or Tyr, more preferably Ser. More preferably Z is —NH₂.

According to an especially preferred aspect, especially preferred compounds include those of formula (I) wherein: Xaa₁ is His or Arg; Xaa₂ is Gly; Xaa₃ is Asp or Glu; Xaa₄ is Phe or napthylalanine; Xaa₅ is Thr or Ser; Xaa₆ is Ser or Thr; Xaa₇ is Asp or Glu; Xaa₈ is Leu or pentylglycine; Xaa₉ is Leu or pentylglycine; Xaa₁₀ is Phe or naphthylalanine; Xaa₁₁ is Ile, Val or t-butyltylglycine; Xaa₁₂ is Glu or Asp; Xaa₁₃ is Trp or Phe; Xaa₁₄, Xaa₁₅, Xaa₁₆, and Xaa₁₇ are independently Pro, homoproline, thioproline, or N-methylalanine; Xaa₁₈ is Ser or Tyr: and Z is —OH or —NH₂; with the proviso that the compound does not have the formula of either SEQ. ID. NOS. 1 or 2. More preferably, Z is —NH₂. Especially preferred compounds include those having the amino acid sequence of SEQ. ID. NOS. 9, 10, 21, 22, 23, 26, 28, 34, 35 and 39.

According to an especially preferred aspect, provided are compounds where Xaa₉ is Leu, Ile, Val or pentylglycine, more preferably Leu or pentylglycine, and Xaa₁₃ is Phe, Tyr or naphthylalanine, more preferably Phe or naphthylalanine. These compounds will exhibit advantageous duration of action and be less subject to oxidative degradation, both in vitro and in vivo, as well as during synthesis of the compound.

Exendin agonist compounds also include those described in International Application No. PCT/US98/24210, filed Nov. 13, 1998, entitled, “Novel Exendin Agonist compounds,” including compounds of the formula (II) [SEQ ID NO. 4]:

Xaa₁ Xaa₂ Xaa₃ Gly Xaa₅ Xaa₆ Xaa₇ Xaa₈ Xaa₉ Xaa₁₀

Xaa₁₁ Xaa₁₂ Xaa₁₃ Xaa₁₄ Xaa₁₅ Xaa₁₆ Xaa₁₇ Ala Xaa₁₉ Xaa₂₀

Xaa₂₁ Xaa₂₂ Xaa₂₃ Xaa₂₄ Xaa₂₅ Xaa₂₆ Xaa₂₇ Xaa₂₈-Z₁; wherein

Xaa₁ is His, Arg or Tyr;

Xaa₂ is Ser, Gly, Ala or Thr;

Xaa₃ is Asp or Glu;

Xaa₅ is Ala or Thr;

Xaa₆ is Ala, Phe, Tyr or naphthylalanine;

Xaa₇ is Thr or Ser;

Xaa₈ is Ala, Ser or Thr;

Xaa₉ is Asp or Glu;

Xaa₁₀ is Ala, Leu, Ile, Val, pentylglycine or Met;

Xaa₁₁ is Ala or Ser;

Xaa₁₂ is Ala or Lys;

Xaa₁₃ is Ala or Gln;

Xaa₁₄ is Ala, Leu, Ile, pentylglycine, Val or Met;

Xaa₁₅ is Ala or Glu;

Xaa₁₆ is Ala or Glu;

Xaa₁₇ is Ala or Glu;

Xaa₁₉ is Ala or Val;

Xaa₂₀ is Ala or Arg;

Xaa₂₁ is Ala or Leu;

Xaa₂₂ is Ala, Phe, Tyr or naphthylalanine;

Xaa₂₃ is Ile, Val, Leu, pentylglycine, tert-butylglycine or Met;

Xaa₂₄ is Ala, Glu or Asp;

Xaa₂₅ is Ala, Trp, Phe, Tyr or naphthylalanine;

Xaa₂₆ is Ala or Leu;

Xaa₂₇ is Ala or Lys;

Xaa₂₈ is Ala or Asn;

Z, is —OH,

—NH₂

Gly-Z₂,

Gly Gly-Z₂,

Gly Gly Xaa₃₁-Z₂,

Gly Gly Xaa₃₁ Ser-Z₂,

Gly Gly Xaa₃₁ Ser Ser-Z₂,

Gly Gly Xaa₃₁ Ser Ser Gly-Z₂,

Gly Gly Xaa₃₁ Ser Ser Gly Ala-Z₂,

Gly Gly Xaa₃₁ Ser Ser Gly Ala Xaa₃₆-Z₂,

Gly Gly Xaa₃₁ Ser Ser Gly Ala Xaa₃₆ Xaa₃₇-Z₂ or

Gly Gly Xaa₃₁ Ser Ser Gly Ala Xaa₃₆ Xaa₃₇ Xaa₃₈-Z₂;

Xaa₃₁, Xaa₃₆, Xaa₃₇ and Xaa₃₈ are independently Pro, homoproline, 3Hyp, 4Hyp, thioproline, N-alkylglycine, N-alkylpentylglycine or N-alkylalanine; and

Z₂ is —OH or —NH₂;

provided that no more than three of Xaa₃, Xaa₅, Xaa₆, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala.

Preferred N-alkyl groups for N-alkylglycine, N-alkylpentylglycine and N-alkylalanine include lower alkyl groups preferably of 1 to about 6 carbon atoms, more preferably of 1 to 4 carbon atoms.

Preferred exendin agonist compounds include those wherein Xaa₁ is His or Tyr. More preferably Xaa₁ is His.

Preferred are those compounds wherein Xaa₂ is Gly.

Preferred are those compounds wherein Xaa₁₄ is Leu, pentylglycine or Met.

Preferred compounds are those wherein Xaa₂₅ is Trp or Phe.

Preferred compounds are those where Xaa₆ is Phe or naphthylalanine; Xaa₂₂ is Phe or naphthylalanine and Xaa₂₃ is Ile or Val.

Preferred are compounds wherein Xaa₃₁, Xaa₃₆, Xaa₃₇ and Xaa₃₈ are independently selected from Pro, homoproline, thioproline and N-alkylalanine.

Preferably Z₁ is —NH₂.

Preferable Z₂ is —NH₂.

According to one aspect, preferred are compounds of formula (II) wherein Xaa₁ is His or Tyr, more preferably His; Xaa₂ is Gly; Xaa₆ is Phe or naphthylalanine; Xaa₁₄ is Leu, pentylglycine or Met; Xaa₂₂ is Phe or naphthylalanine; Xaa₂₃ is Ile or Val; Xaa₃₁, Xaa₃₆, Xaa₃₇ and Xaa₃₈ are independently selected from Pro, homoproline, thioproline or N-alkylalanine. More preferably Z₁ is —NH₂.

According to an especially preferred aspect, especially preferred compounds include those of formula (II) wherein: Xaa₁ is His or Arg; Xaa₂ is Gly or Ala; Xaa₃ is Asp or Glu; Xaa₅ is Ala or Thr; Xaa₆ is Ala, Phe or nephthylalaine; Xaa₇ is Thr or Ser; Xaa₈ is Ala, Ser or Thr; Xaa₉ is Asp or Glu; Xaa₁₀ is Ala, Leu or pentylglycine; Xaa₁₁ is Ala or Ser; Xaa₁₂ is Ala or Lys; Xaa₁₃ is Ala or Gln; Xaa₁₄ is Ala, Leu or pentylglycine; Xaa₁₅ is Ala or Glu; Xaa₁₆ is Ala or Glu; Xaa₁₇ is Ala or Glu; Xaa₁₉ is Ala or Val; Xaa₂₀ is Ala or Arg; Xaa₂₁ is Ala or Leu; Xaa₂₂ is Phe or naphthylalanine; Xaa₂₃ is Ile, Val or tert-butylglycine; Xaa₂₄ is Ala, Glu or Asp; Xaa₂₅ is Ala, Trp or Phe; Xaa₂₆ is Ala or Leu; Xaa₂₇ is Ala or Lys; Xaa₂₈ is Ala or Asn; Z, is —OH, —NH₂, Gly-Z₂, Gly Gly-Z₂, Gly Gly Xaa₃₁-Z₂, Gly Gly Xaa₃₁ Ser-Z₂, Gly Gly Xaa₃₁ Ser Ser-Z₂, Gly Gly Xaa₃₁ Ser Ser Gly-Z₂, Gly Gly Xaa₃₁ Ser Ser Gly Ala-Z₂, Gly Gly Xaa₃₁ Ser Ser Gly Ala Xaa₃₆-Z₂, Gly Gly Xaa₃₁ Ser Ser Gly Ala Xaa₃₆ Xaa₃₇-Z₂, Gly Gly Xaa₃₁ Ser Ser Gly Ala Xaa₃₆ Xaa₃₇ Xaa₃₈-Z₂; Xaa₃₁, Xaa₃₆, Xaa₃₇ and Xaa₃₈ being independently Pro homoproline, thioproline or N-methylalanine; and Z₂ being —OH or —NH₂; provided that no more than three of Xaa₃, Xaa₅, Xaa₆, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala. Especially preferred compounds include those having the amino acid sequence of SEQ. ID. NOS. 40-61.

According to an especially preferred aspect, provided are compounds where Xaa₁₄ is Leu, Ile, Val or pentylglycine, more preferably Leu or pentylglycine, and Xaa₂₅ is Phe, Tyr or naphthylalanine, more preferably Phe or naphthylalanine. These compounds will be less susceptive to oxidative degration, both in vitro and in vivo, as well as during synthesis of the compound.

Exendin agonist compounds also include those described in International Patent Application No. PCT/US98/24273, filed Nov. 13, 1998, entitled, “Novel Exendin Agonist Compounds,” including compounds of the formula (III) [SEQ ID NO. 5]:

Xaa₁ Xaa₂ Xaa₃ Xaa₄ Xaa₅ Xaa₆ Xaa₇ Xaa₈ Xaa₉ Xaa₁₀

Xaa₁₁ Xaa₁₂ Xaa₁₃ Xaa₁₄ Xaa₁₅ Xaa₁₆ Xaa₁₇ Ala Xaa₁₉ Xaa₂₀

Xaa₂l Xaa₂₂Xaa₂₃ Xaa₂₄ Xaa₂₅ Xaa₂₆ Xaa₂₇ Xaa₂₈-Z₁; wherein

Xaa₁ is His, Arg, Tyr, Ala, Norval, Val, or Norleu;

Xaa₂ is Ser, Gly, Ala or Thr;

Xaa₃ is Ala, Asp or Glu;

Xaa₄ is Ala, Norval, Val, Norleu or Gly;

Xaa₅ is Ala or Thr;

Xaa₆ is Phe, Tyr or naphthylalanine;

Xaa₇ is Thr or Ser;

Xaa₈ is Ala, Ser or Thr;

Xaa₉ is Ala, Norval, Val, Norleu, Asp or Glu;

Xaa₁₀ is Ala, Leu, Ile, Val, pentylglycine or Met;

Xaa₁₁ is Ala or Ser;

Xaa₁₂ is Ala or Lys;

Xaa₁₃ is Ala or Gln;

Xaa₁₄ is Ala, Leu, Ile, pentylglycine, Val or Met;

Xaa₁₅ is Ala or Glu;

Xaa₁₆ is Ala or Glu;

Xaa₁₇ is Ala or Glu;

Xaa₁₉ is Ala or Val;

Xaa₂₀ is Ala or Arg;

Xaa₂₁ is Ala or Leu;

Xaa₂₂ is Phe, Tyr or naphthylalanine;

Xaa₂₃ is Ile, Val, Leu, pentylglycine, tert-butylglycine or Met;

Xaa₂₄ is Ala, Glu or Asp;

Xaa₂₅ is Ala, Trp, Phe, Tyr or naphthylalanine;

Xaa₂₆ is Ala or Leu;

Xaa₂₇ is Ala or Lys;

Xaa₂₈ is Ala or Asn;

Z₁ is —OH,

—NH₂,

Gly-Z₂,

Gly Gly-Z₂,

Gly Gly Xaa₃₁-Z₂,

Gly Gly Xaa₃₁ Ser-Z₂,

Gly Gly Xaa₃₁ Ser Ser-Z₂,

Gly Gly Xaa₃₁ Ser Ser Gly-Z₂,

Gly Gly Xaa₃₁ Ser Ser Gly Ala-Z₂,

Gly Gly Xaa₃₁ Ser Ser Gly Ala Xaa₃₆-Z₂,

Gly Gly Xaa₃₁ Ser Ser Gly Ala Xaa₃₆ Xaa₃₇-Z₂,

Gly Gly Xaa₃₁ Ser Ser Gly Ala Xaa₃₆ Xaa₃₇ Xaa₃₈-Z₂ or Gly Gly Xaa₃₁ Ser Ser Gly Ala Xaa₃₆ Xaa₃₇ Xaa₃₈ Xaa₃₉-Z₂;

Wherein Xaa₃₁, Xaa₃₆, Xaa₃₇ and Xaa₃₈ are independently

Pro, homoproline, 3Hyp, 4Hyp, thioproline, N-alkylglycine, N-alkylpentylglycine or N-alkylalanine;

Xaa₃₉ is Ser, Thr, Lys or Ala; and

Z₂ is —OH or —NH₂;

provided that no more than three of Xaa₃, Xaa₄, Xaa₅, Xaa₆, Xaa₈, Xaa₉, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala; and provided also that, if Xaa₁ is His, Arg or Tyr, then at least one of Xaa₃, Xaa₄ and Xaa₉ is Ala.

Compounds useful in the formulations of the invention also include glucagon-like peptide 1 and analogs and agonists thereof. Such compounds are known in the art and include, for example, those disclosed in WO 8706941, WO 0198331, and WO 9808871.

Additional compounds useful in the formulations of the invention include those disclosed in the sequence listing appended hereto (including SEQ ID Nos 61-188).

Preparation of Compounds

The peptide compounds that constitute active ingredients of the formulations and dosages of the present invention (e.g., exendins, exendin agonists and antagonists, and exendin analogs) may be prepared using any method, for example recombinant or standard solid-phase peptide synthesis techniques and preferably an automated or semiautomated peptide synthesizer. An example of the preparation of exendin-3 and exendin-4 is described in Examples 1 and 2 below. The preparation of additional exendin agonist peptide analogs is described in, for example, WO 0041546.

Typically, using automated or semiautomated peptide synthesis techniques, an α-N-carbamoyl protected amino acid and an amino acid attached to the growing peptide chain on a resin are coupled at room temperature in an inert solvent such as dimethylformamide, N-methylpyrrolidinone or methylene chloride in the presence of coupling agents such as dicyclohexylcarbodiimide and 1-hydroxybenzotriazole in the presence of a base such as diisopropylethylamine. The α-N-carbamoyl protecting group is removed from the resulting peptide-resin using a reagent such as trifluoroacetic acid or piperidine, and the coupling reaction repeated with the next desired N-protected amino acid to be added to the peptide chain. Suitable N-protecting groups are well known in the art, with t-butyloxycarbonyl (tBoc) and fluorenylmethoxycarbonyl (Fmoc) being preferred herein.

The solvents, amino acid derivatives and 4-methylbenzhydryl-amine resin used in the peptide synthesizer may be purchased from Applied Biosystems Inc. (Foster City, Calif.). The following side-chain protected amino acids may be purchased from Applied Biosystems, Inc.: Boc-Arg(Mts), Fmoc-Arg(Pmc), Boc-Thr(Bzl), Fmoc-Thr(t-Bu), Boc-Ser(Bzl), Fmoc-Ser(t-Bu), Boc-Tyr(BrZ), Fmoc-Tyr(t-Bu), Boc-Lys(Cl-Z), Fmoc-Lys(Boc), Boc-Glu(Bzl), Fmoc-Glu(t-Bu), Fmoc-His(Trt), Fmoc-Asn(Trt), and Fmoc-Gln(Trt). Boc-His(BOM) may be purchased from Applied Biosystems, Inc. or Bachem Inc. (Torrance, Calif.). Anisole, dimethylsulfide, phenol, ethanedithiol, and thioanisole may be obtained from Aldrich Chemical Company (Milwaukee, Wis.). Air Products and Chemicals (Allentown, Pa.) supplies HF. Ethyl ether, acetic acid, and methanol may be purchased from Fisher Scientific (Pittsburgh, Pa.).

Solid phase peptide synthesis may be carried out with an automatic peptide synthesizer (Model 430A, Applied Biosystems Inc., Foster City, Calif.) using the NMP/HOBt (Option 1) system and tBoc or Fmoc chemistry (see, Applied Biosystems User's Manual for the ABI 430A Peptide Synthesizer, Version 1.3B Jul. 1, 1988, section 6, pp. 49-70, Applied Biosystems, Inc., Foster City, Calif.) with capping. Boc-peptide-resins may be cleaved with HF (−5° C. to 0° C., 1 hour). The peptide may be extracted from the resin with alternating water and acetic acid, and the filtrates lyophilized. The Fmoc-peptide resins may be cleaved according to standard methods (Introduction to Cleavage Techniques, Applied Biosystems, Inc., 1990, pp. 6-12). Peptides may also be assembled using an Advanced Chem Tech Synthesizer (Model MPS 350, Louisville, Ky.).

Peptides may be purified by RP-HPLC (preparative and analytical) using a Waters Delta Prep 3000 system. A C4, C8 or C18 preparative column (10 μ, 2.2×25 cm; Vydac, Hesperia, Calif.) may be used to isolate peptides, and purity may be determined using a C4, C8 or C18 analytical column (5 μ, 0.46×25 cm; Vydac). Solvents (A=0.1% TFA/water and B=0.1% TFA/CH₃CN) may be delivered to the analytical column at a flow rate of 1.0 ml/min and to the preparative column at 15 ml/min. Amino acid analyses may be performed on the Waters Pico Tag system and processed using the Maxima program. Peptides may be hydrolyzed by vapor-phase acid hydrolysis (115° C., 20-24 h). Hydrolysates may be derivatized and analyzed by standard methods (Cohen, et al, The Pico Tag Method: A Manual of Advanced Techniques for Amino Acid Analysis, pp. 11-52, Millipore Corporation, Milford, MA (1989)). Fast atom bombardment analysis may be carried out by M-Scan, Incorporated (West Chester, Pa.). Mass i:.e calibration may be performed using cesium iodide or cesium iodide/glycerol. Plasma desorption ionization analysis using time of flight detection may be carried out on an Applied Biosystems Bio-Ion 20 mass spectrometer. Electrospray mass spectroscopy may be carried and on a VG-Trio machine.

Peptide active ingredient compounds useful in the formulations and dosages of the invention may also be prepared using recombinant DNA techniques, using methods now known in the art. See, eg., Sambrook et al., Molecular Cloning: A Laboratory Manual, 2d Ed., Cold Spring Harbor (1989).

Utility

The formulations and dosages described herein are useful in view of their pharmacological properties. In particular, the formulations and dosages of the invention are effective as exendins and exendin agonists, and possess activity as agents to lower blood glucose, to regulate gastric motility and to slow gastric emptying and reduce food intake.

Formulation and Administration

Exendins, exendin agonists and antagonists, exendin analogs, formulations and dosages of the invention are useful in view of their exendin-like or anti-exendin effects, and may conveniently be provided in the form of formulations suitable for parenteral (including intravenous, intradermal, intraperitoneal, intramuscular and subcutaneous) administration. Also described herein are formulations and dosages useful in alternative delivery routes, including oral, nasal, buccal, sublingual, intra-tracheal, transdermal, transmucosal, and pulmonary.

Other suitable means of delivering exendin and exendin analogs include subcutaneous, intradermal, intravenous, intraperitoneal and intramuscular injections, oral, sublingual, intratracheal, pulmonary, nasal, buccal, transdermal and transmucosal gel or suppository. Because bioavailability of various formulations varies, plasma levels can be used to determine appropriate dosing. For exendin-4, for example, a target circulating plasma concentration range of between about 5 pg/ml and about 5000 pg/ml is preferred, more preferably between about 5 pg/ml and about 500 pg/ml, most preferably between about 10 pg/ml and about 200 pg/ml. For exendin agonists and analogs, adjustments based on potency of the agonist or analog, relative to exendin, are appropriate and within the skill in the art.

Compounds useful in the invention can be provided as parenteral compositions for injection, infusion, or implant. They can be provided for ingestion, absorption, etc., and may be liquid, solid, semi-solid, gel, or in any suitable matrix or carrier. Generally, they can, for example, be suspended in an inert oil, such as vegetable oil such as sesame, peanut, olive oil, or -other acceptable carrier. Preferably, they are suspended or dissolved in an aqueous carrier, for example, in an isotonic buffer solution at a pH of about 3.0 to about 8.0, more specifically from about 4.0 to 6.0, and preferably from about 4.0 to about 5.0. These compositions may be sterilized by conventional sterilization techniques, or may be sterile filtered. The compositions may contain pharmaceutically acceptable auxiliary substances as required to approximate physiological conditions, such as pH buffering agents. Useful buffers include for example, sodium acetate/acetic acid buffers. The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions.

The exendin and exendin agonist compounds can also be formulated as pharmaceutically acceptable salts (e.g., acid addition salts) and/or complexes thereof. Pharmaceutically acceptable salts are non-toxic salts at the concentration at which they are administered. The preparation of such salts can facilitate the pharmacological use by altering the physical-chemical characteristics of the composition without preventing the composition from exerting its physiological effect. Examples of useful alterations in physical properties include lowering the melting point to facilitate transmucosal administration and increasing the solubility to facilitate the administration of higher concentrations of the drug.

Pharmaceutically acceptable salts include acid addition salts such as those containing sulfate, hydrochloride, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethane sulfonic acid, benzene sulfonic acid, p-toluenesulfonic acid, cyclohexyl sulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

Generally, carriers or excipients known in the art can also be used to facilitate administration of the dosages of the present invention. Examples of carriers and excipients include calcium carbonate, calcium phosphate, various sugars such as lactose, or types of starch, cellulose derivatives, gelatin, vegetable oils, polyethylene glycols and physiologically compatible solvents.

If desired, solutions of the above dosage compositions may be thickened with a thickening agent such as methylcellulose. They may be prepared in emulsified form, such as either water in oil or oil in water. Any of a wide variety of pharmaceutically acceptable emulsifying agents may be employed including, for example, acacia powder, a non-ionic surfactant (such as a Tween), or an ionic surfactant (such as alkali polyether alcohol sulfates or sulfonates, eg., a Triton).

In general, formulations and dosage compositions of the invention are prepared by mixing the ingredients following generally accepted procedures. For example, the selected components may be simply mixed in a blender or other standard device to produce a concentrated mixture which may then be adjusted to the final concentration and viscosity by the addition of water or thickening agent and possibly a buffer to control pH or an additional solute to control tonicity.

Other pharmaceutically acceptable carriers and their formulation are described in standard formulation treatises, eg, Remington's Pharmaceutical Sciences by E. W. Martin. See also Wang, Y. J. and Hanson, M. A. “Parenteral Formulations of Proteins and Peptides: Stability and Stabilizers,” Journal of Parenteral Science and Technology, Technical Report No. 10, Supp. 42:2S (1988).

For use by the physician, the compounds will be provided in dosage unit form containing an amount of an exendin agonist, with or without another therapeutic agent, for example, a glucose-lowering agent, a gastric emptying modulating agent, a lipid lowering agent, or a food intake inhibitor agent. Therapeutically effective amounts of an exendin agonist for use in the control of blood glucose or in the control of gastric emptying and in conditions in which gastric emptying is beneficially slowed or regulated are those that decrease post-prandial blood glucose levels, preferably to no more than about 8 or 9 mM or such that blood glucose levels are reduced as desired. In diabetic or glucose intolerant individuals, plasma glucose levels are higher than in normal individuals. In such individuals, beneficial reduction or “smoothing” of post-prandial blood glucose levels may be obtained. As will be recognized by those in the field, an effective amount of therapeutic agent will vary with many factors including the patient's physical condition, the blood sugar level or level of inhibition of gastric emptying to be obtained, or the desired level of food intake reduction, and other factors.

Such pharmaceutical compositions are useful in causing increased insulin sensitivity in a subject and may be used as well in disorders, such as diabetes, where sensitivity to insulin is beneficially increased.

The effective daily doses of the compounds are described. The exact dose to be administered may be determined by the attending clinician and may be further dependent upon the efficacy of the particular exendin or exendin agonist compound used, as well as upon the age, weight and condition of the individual. A preferred means of delivering the compounds described is to administer them using a controlled release formulation (e.g., injectable or implantable) that slowly releases the compound over periods of hours to months. One advantage of this mode of administration is improvement in patient compliance, since daily or multiple daily doses may be missed by the patient.

The optimal mode of administration of compounds of the present application to a patient depend on factors known in the art such as the particular disease or disorder, the desired effect, and the type of patient. While the compounds will typically be used to treat human patients, they may also be used to treat similar or identical diseases in other vertebrates such as other primates, farm animals such as swine, cattle and poultry, and sports animals and pets such as horses, dogs and cats.

The invention includes liquid formulations of exendins and exendin agonists that comprise an exendin or exendin agonist mixed together with a buffer (preferably an acetate buffer), an iso-osmolality modifier (preferably mannitol), and optionally containing a preservative (preferably m-cresol), the formulation having a pH of between about 3.0 and about 8.0 (preferably between about 4.0 and about 5.0). Other pH ranges may be preferable for different analogs based on their chemical characteristics.

The formulation which best supports a parenteral liquid dosage form is one in which the active ingredient(s) is stable with adequate buffering capacity to maintain the pH of the solution over the intended shelf life of the product. The dosage form should be either an isotonic and/or an iso-osmolar solution to either facilitate stability of the active ingredient or lessen the pain on injection or both. Devices that deliver very small injection volumes, however, may not require that the formulation be either isotonic and/or iso-osmolar. If the dosage form is packaged as a unit-dose, then a preservative may be included but is not required. If, however, the dosage form is packaged in a multi-use container, then a preservative is necessary.

For compounds having exendin-4-like potency, these dosage forms preferably include approximately 0.005 to about 5%, more specifically from about 0.005 to about 1.0%, or from about 0.005 to about 0.05% (w/v), respectively of the active ingredient in an aqueous system along with approximately 0.02 to 0.5% (w/v) of an acetate, phosphate, citrate or glutamate or similar buffer either alone or in combination to obtain a pH of the final composition of approximately 3.0 to 7.0, more specifically from about pH 4.0 to about 6.0, or from about 4.0 to 5.0, as well as either approximately 1.0 to 10% (w/v) of a carbohydrate or polyhydric alcohol iso-osmolality modifier (preferably mannitol) or up to about 0.9% saline or a combination of both leading to an isotonic or an iso-osmolar solution in an aqueous continuous phase. Approximately 0.005 to 1.0% (w/v) of an anti-microbial preservative selected from the group consisting of m-cresol, benzyl alcohol, methyl ethyl, propyl and butyl parabens and phenol is also present if the formulation is packaged in a multi-use container. A sufficient amount of water for injection is added to obtain the desired concentration of solution. Sodium chloride, as well as other excipients, may also be present, if desired. Such excipients, however, must maintain the overall stability of the active ingredient.

Polyhydric alcohols and carbohydrates share the same feature in their backbones, i.e., —CHOH—CHOH—. The polyhydric alcohols include such compounds as sorbitol, mannitol, glycerol, and polyethylene glycols (PEGs). These compounds are straight-chain molecules. The carbohydrates, such as mannose, ribose, trehalose, maltose, glycerol, inositol, glucose and lactose, on the other hand, are cyclic molecules that may contain a keto or aldehyde group. These two classes of compounds will also be effective in stabilizing protein against denaturation caused by elevated temperature and by freeze-thaw or freeze-drying processes. Suitable carbohydrates include galactose, arabinose, lactose or any other carbohydrate which does not have an adverse affect on a diabetic patient, i.e., the carbohydrate is not metabolized to form large concentrations of glucose in the blood. Such carbohydrates are well known in the art as suitable for diabetics.

Preferably, the peptides of the present invention are admixed with a polyhydric alcohol such as sorbitol, mannitol, inositol, glycerol, xylitol, and polypropylene/ethylene glycol copolymer, as well as various polyethylene glycols (PEG) of molecular weight 200, 400, 1450, 3350, 4000, 6000, and 8000). Mannitol is the preferred polyhydric alcohol. The liquid formulation of the invention should be substantially isotonic and/or iso-osmolar. An isotonic solution may be defined as a solution that has a concentration of electrolytes, or a combination of electrolytes and non-electrolytes that will exert equivalent osmotic pressure as that into which it is being introduced, here for example in the case of parenteral injection of the formulation, a mammalian tissue. Similarly, an iso-osmolar solution may be defined as a solution that has a concentration of non-electrolytes that will exert equivalent osmotic pressure as that into which it is being introduced. As used herein, “substantially isotonic” means within ±20% of isotonicity, preferably within ±10%. As used herein, “substantially iso-osmolar” means within ±20% of iso-osmolality, preferably within ±10%. The formulated product for injection is included within a container, typically, for example, a vial, cartridge, prefilled syringe or disposable pen.

The formulation which best supports a unit-dose parenteral lyophilized dosage form is one in which the active ingredient is reasonably stable, with or without adequate buffering capacity to maintain the pH of the solution over the intended shelf life of the reconstituted product. The dosage form should contain a bulking agent to facilitate cake formation. The bulking agent may also act as a tonicifer and/or iso-osmolality modifier upon reconstitution to either facilitate stability of the active ingredient and/or lessen the pain on injection. As noted above, devices that deliver very small injection volumes may not require the formulation to be isotonic and/or iso-osmolar. A surfactant may also benefit the properties of the cake and/or facilitate reconstitution.

These dosage forms include approximately 0.005 to about 5%, more specifically from about 0.005 to about 0.02%, or 0.005 to 0.05% (w/v) of the active ingredient if it is similar to exendin 4 in potency. It may not be necessary to include a buffer in the formulation and/or to reconstitute the lyophile with a buffer if the intention is to consume the contents of the container within the stability period established for the reconstituted active ingredient. If a buffer is used, it may be included in the lyophile or in the reconstitution solvent. Therefore, the formulation and/or the reconstitution solvent may contain individually or collectively approximately 0.02 to 0.5% (w/v) of an acetate, phosphate, citrate or glutamate buffer either alone or in combination to obtain a pH of the final composition of approximately 3.0 to 7.0, more specifically from about pH 4.0 to about 6.0, or from about 4.0 to 5.0. The bulking agent may consist of either approximately 1.0 to 10% (w/v) of a carbohydrate or polyhydric alcohol iso-osmolality modifier (as described above) or up to 0.9% saline or a combination of both leading to a isotonic or iso-osmolar solution in the reconstituted aqueous phase. A surfactant, preferably about 0.1 to about 1.0% (w/v) of polysorbate 80 or other non-ionic detergent, may be included. As noted above, sodium chloride, as well as other excipients, may also be present in the lyophilized unit-dosage formulation, if desired. Such excipients, however, must maintain the overall stability of the active ingredient. The formulation will be lyophilized within the validation parameters identified A) to maintain stability of the active ingredient.

The liquid formulation of the invention before lyophilization should be substantially isotonic and/or iso-osmolar either before lyophilization or to enable formation of isotonic and/or iso-osmolar solutions after reconstitution if isotonicity is desired (e.g., for infusion or injection formulations). The formulation should be used within the period established by shelf-life studies on both the lyophilized form and following reconstitution. The lyophilized product is included within a container, typically, for example, a vial. If other containers are used such as a cartridge, pre-filled syringe, or disposable pen, the reconstitution solvent may also be included.

As with the parenteral liquid and lyophilized unit-dosage formulations described above, the formulation which best supports a multi-dose parenteral lyophilized dosage form is one in which the active ingredient is reasonably stable with adequate buffering capacity to maintain the pH of the solution over the intended “in-use” shelf-life of the product. The dosage form should contain a bulking agent to facilitate cake formation. The bulking agent may also act as a tonicifer and/or iso-osmolality modifier upon reconstitution to either facilitate stability of the active ingredient or lessen the pain on injection or both. Again, devices that deliver very small injection volumes may not require the formulation to be either isotonic and/or iso-osmolar. A preservative is, however, necessary to facilitate multiple use by the patient.

It may not be necessary to include a buffer in the formulation and/or to reconstitute the lyophile with a buffer if the intention is to consume the contents of the container within the stability period established for the reconstituted active ingredient. If a buffer is used, it may be included in the lyophile or in the reconstitution solvent. Therefore, the formulation and/or the reconstitution solvent may contain individually or collectively approximately 0.02 to 0.5% (w/v) of an acetate, phosphate, citrate or glutamate buffer either alone or in combination to obtain a pH of the final composition of approximately 3.0 to 8.0, more specifically from about pH 4.0 to about 6.0, or from about 4.0 to 5.0. The bulking agent may consist of either approximately 1.0 to 10% (w/v) of a carbohydrate or a polyhydric alcohol iso-osmolality modifier (preferably mannitol) or up to 0.9% saline, or a combination of both, leading to an isotonic or iso-osmolar solution in the reconstituted aqueous phase. A surfactant, preferably about 0.1 to about 1.0% (w/v) of polysorbate 80 or other non-ionic detergent, may be included. Approximately 0.005 to 1.0% (w/v) of an anti-microbial preservative selected from the group consisting of m-cresol, benzyl alcohol, methyl, ethyl, propyl and butyl parabens and phenol (preferably m-cresol) is also present if the formulation is packaged in a multi-use container. Sodium chloride, as well as other excipients, may also be present, if desired.

A preferred formulation of the invention is a liquid, solid, or semi-solid depot, slow, or continuous release formulation capable of delivering an active ingredient of the invention over a time period of at least one hour. In preferred embodiments, the release occurs over a period of 24 hours to four months. Such slow or extended release formulations preferably consist of the active ingredient in a slow dissolving form or formulation, such as a slow-dissolving peptide crystal (such as disclosed in, for example, U.S. Pat. No. 6,380,357), in a matrix, or in a coating such as, e.g., an enteric coating or slow-disolving coating (e.g., coated granules of active ingredient). Slow release matrices are commonly a biodegradable polymer, non-biodegradable polymer, wax, fatty material, etc., and are known in the art (e.g., see U.S. Pat. Nos. 6,368,630 and related patents, 6,379,704 and related patents). In addition, parenteral controlled release delivery can be achieved by forming polymeric microcapsules, matrices, solutions, implants and devices and administering them parenterally or by surgical means. These dosage forms would typically have a lower bioavailability due to entrapment of some of the peptide in the polymer matrix or device. (See e.g., U.S. Pat. Nos. 6,379,704, 6,379,703, and 6,296,842).

The invention further includes solid or semi-solid forms useful for oral, buccal, sublingual, intra-tracheal, nasal, and pulmonary delivery. The formulations that best support pulmonary and/or intra-tracheal dosage forms may be either preserved or unpreserved liquid formulations and/or dry powder formulations. The preserved or unpreserved liquid formulations will be essentially identical to the formulations described above under preserved or unpreserved liquid parenteral formulations. For exendin for example, the pH of the solution is preferably about 3.0 to 7.0, more preferably from about 4.0 to 6.0, or from about 4.0 to 5.0, with a pH greater than or equal to about 5.0 being most preferred to reduce the potential for bronchoconstriction. The dry powder formulations and solid dosage forms (oral, sublingual and buccal) may contain a bulking agent and/or salts to facilitate particle size formation and appropriate particle size distribution. A surfactant and/or salts may also benefit the properties of the particle morphology and/or facilitate tissue uptake of the active ingredient.

Dry powder and solid dosage forms can contain active ingredient in a range from 1% to 100% (w/w), respectively. It may not be necessary to include a bulking agent and/or salts to facilitate particle size formation and/or distribution. The bulking agent and/or salts may consist of either approximately 0 to 99% (w/w) of a carbohydrate or polyhydric alcohol or approximately 0 to 99% salt or a combination of both leading to the preferred particle size and distribution. A surfactant, preferably about 0.1 to about 1.0% (w/w) of polysorbate 80 or other non-ionic detergent, may be included. Sodium chloride, as well as other excipients, may also be present, if desired. Such excipients, however, will maintain the overall stability of the active ingredient and facilitate the proper level of hydration or dissolution after administration. Typically, some formulations include a enzyme inhibitor, penetration enhancer or complexing agent to facilitate absorption from the site of administration. In solid dosage forms, excipients typically known in the art are incorporated and some forms may include coatings to protect the peptide from the biological environment following administration.

The formulations that best support nasal and/or intra-tracheal dosage forms may be either preserved or unpreserved liquid dosage formulations or dry powder formulations as mentioned earlier. Ingredients to facilitate absorption through mucosal barriers, such as ethanol or propylene glycol, and to inhibit enzymes that degrade the peptide may be added.

Atomized liquids, dissolvable gels, adhesive tablets and/or patches may be used to facilitate buccal delivery. For example, the gels may be prepared from various types of starch and/or cellulose derivatives. Ingredients to facilitate absorption through mucosal barriers, such as ethanol or propylene glycol, may be added.

Sublingual delivery may be best supported solid dosage forms that may be similar to oral solid dosage forms except that they must be readily dissolvable under the tongue.

Oral delivery may be best supported by a liquid (gel cap) formulation that is similar to the parenteral liquid formulation except that the solution does not contain a preservative, may be more concentrated, or may consist of a suspension and may contain additional additives to facilitate uptake of the active ingredient or inhibit degradation in the alimentary canal . Solid dosage forms will contain excipients know in the art along with the active ingredient to facilitate tablet formation. These ingredients may include polyhedral alcohols (such as mannitol), carbohydrates, or types of starch, cellulose derivatives, and/or other inert, physiologically compatible materials. The tablet may be coated to minimize digestion in the stomach and thereby facilitate dissolution and uptake further along the alimentary canal.

Further within the scope of the invention are preferred dosages for exendins and exendin agonists when given by injection, and when given by other routes. Thus, formulations for exendin and exendin agonists having comparable potency are provided. For administration (e.g., by injection, infusion, slow release, ingestion, etc.), doses will generally be from about 0.5 μg to about 1000 μg, preferably falling into the range of about 1.0 μg/day to about 500 μg/day, generally in the range of about 0.001 to about 1.0 μg per kilogram, for example given one to four times per day or as a continuous infusion or release. Typically, for the patient with diabetes who weighs in the range from about 70 kilograms (average for the type 1 diabetic) to about 90 kilograms (average for the type 2 diabetic), for example, this will result in the total administration of about 1.0 to about 120 μg per day in continuous, single or divided doses. If administered in divided doses, the doses are preferably administered two or four times per day, more preferably two times per day.

Preferably, the exendin or exendin agonist is administered parenterally using a solution, preferably by injection, for example, by subcutaneous injection. Preferably, about 1 μg-30 μg to about 1 mg of the exendin or exendin agonist is administered per day for such a formulation. More preferably, about 1-30 μg to about 500 μg, or about 1-30 μg to about 50 μg of the exendin or exendin agonist is administered per day. Most preferably, about 3 μg to about 50 μg of the exendin or exendin agonist is administered per day. Preferred doses based upon patient weight for compounds having approximately the potency of exendin-4 range from about 0.0005 μg/kg per dose or per day to about 2.0 μg/kg per dose or per day. More preferably, doses based upon patient weight for compounds having approximately the potency of exendin-4 range from about 0.02 μg/kg per dose (or per day if continuously administered by e.g., infusion or slow release depot composition) to about 0.1 μg/kg per dose or per day. Most preferably, bolus doses based upon patient weight for compounds having approximately the potency of exendin-4 range from about 0.02 μg/kg per dose to about 0.1 μg/kg per dose. Bolus doses are administered from 1 to 4 times per day, preferably from 1 to 2 times per day. Doses of exendins or exendin agonists will normally be lower if given by continuous infusion, preferably between about 0.0005 μg/kg/day to about 2 μg/kg/day, more preferably between about 0.2 μg/kg/day to about 1.0 μg/kg/day.

Plasma levels resulting from any administrations will achieve therapeutic levels. For bolus doses of compounds with potency comparable to exendin 4, peak plasma levels will preferably generally exceed about 40 pg/ml, more preferably about 100 pg/ml, and for continuous or prolonged release administration (i.e., delivery occurring over about 1 hour to several weeks or months, or longer), peak or average sustained plasma levels will preferably exceed about 5 pg/ml, more preferably about 40 pg/ml. Average sustained plasma levels are determined by taking the average of two or more measurements of plasma levels over the intended duration of exendin or agonist administration. The “intended duration” of the administration is that time over which the therapeutic level of the exendin or agonist is intended to be delivered. For example, a slow release biodegradable formulation implanted once a month may be intended (predetermined) to release therapeutic amounts of drug over a period of one month. Remnants of the formulation may persist for longer than a month, but release drug at sub-therapeutic levels. The average sustained plasma levels would be the average of those exendin plasma levels measured during the intended therapeutic release period of one month.

Doses of exendins or exendin agonists will normally be higher if given by non-injection methods, such as oral, buccal, sublingual, nasal, intratracheal, pulmonary or transdermal or transmucosal delivery.

For example, oral dosages according to the present invention will include from about 10 to about 100 times the active ingredient used in parenteral (e.g., injectable) formulations, e.g., from about 5 to about 12,000 μg per day in single or divided doses, preferably from about 5 to about 5,000 μg per day. Pulmonary dosages according to the present invention will include from about 10 to about 100 times the active ingredient, e.g., from about 1 to about 12,000 μg per day in single or divided doses, preferably about 50 to 1000 μg per day. Nasal, buccal and sublingual dosages according to the present invention will also include from about 10 to about 100 times the active ingredient, e.g., from about 1 to about 12,000 μg per day in single or divided doses.

Preferred dosages for nasal administration are from about 10-1000 to about 1200-12,000 μg per day, for buccal administration from about 10-1000 to about 1200-12,000 μg per day, and for sublingual administration from about 10-1000 to about 1200-8,000 μg per day. Sublingual dosages are preferably smaller than buccal dosages. Administration dosages for exendin agonists having less than or greater than the potency of exendin-4 are increased or decreased as appropriate from those described above and elsewhere herein.

Clinical Studies

Studies of exendin have been conducted in human subjects and serve to demonstrate the utility of exendin and exendin analogs. A summary of selected studies is presented below.

As described in Example 8 below, a double blind, placebo-controlled single ascending dose study examining the safety, tolerability, and pharmacokinetics of subcutaneous exendin-4 in healthy volunteers has been completed. Five single subcutaneous doses of exendin-4 (0.01, 0.05, 0.1, 0.2 or 0.3 μg/kg) were studied in 40 healthy male volunteers in the fasting state. Maximum plasma exendin-4 concentrations were achieved between one and two hours post-dose with little difference among the doses examined. Examination of the data indicated a dose dependent increase for Cₘₐₓ. There were no serious adverse events reported in this study.

In the healthy male volunteers that participated in this study, exendin-4 was well tolerated at subcutaneous doses up to and including 0.1 μg/kg. A decrease in plasma glucose concentration was also observed at this dose. At doses of 0.2 μg/kg and higher, the most commonly observed adverse events were headache, nausea, vomiting, dizziness, and postural hypotension. There was a transient fall in plasma glucose concentration following administration of doses of 0.05 μg/kg and above.

Example 10 below describes a further study of the dose-response relationship for the glucose-lowering effect of exendin-4 at doses less than 0.1 μg/kg. Fourteen subjects [mean (±SE) age 55±2; mean BMI (30.2±1.6 kg/m²)] with type 2 diabetes treated with diet±oral hypoglycemic agents were studied following withdrawal of oral agents for 10-14 days. Assessments were made following randomized, subcutaneous injection of placebo, 0.01, 0.02, 0.05 and 0.1 μg/kg exendin-4 on separate days following an overnight fast. Injections were given immediately before ingestion of a standardized Sustacal® meal (7 kcal/kg) followed by collection of plasma glucose samples at frequent intervals during the subsequent 300 minutes.

The glycemic response was quantified as the time-weighted mean (±SE) change in plasma glucose concentration during the 5-hr period. The response ranged from a +42.0±7.9 mg/dL increment above the fasting glucose concentration for placebo compared to a 30.5±8.6 mg/dL decrement below the fasting glucose concentration with 0.1 μg/kg exendin-4.

The ED₅₀ for this glucose lowering effect was 0.038 μg/kg. Exendin-4 doses less than 0.1 μg/kg appeared to disassociate the glucose lowering effects from the gastrointestinal side effects. Example 10 shows that exendin-4 was not only well tolerated at doses less than 0.1 μg/kg, but that these doses substantially lowered postprandial plasma glucose concentrations (ED₅₀ of 0.038 μg/kg) in people with type 2 diabetes.

Alternate Routes of Delivery

The feasibility of alternate routes of delivery for exendin-4 has been explored by measuring exendin-4 in the circulation of animals in conjunction with observation of a biologic response, such as plasma glucose lowering in diabetic animals, after administration. Passage of exendin-4 has been investigated across several surfaces, the respiratory tract (nasal, tracheal, and pulmonary routes) and the gut (sublingual, gavage and intraduodenal routes). Biologic effect and appearance of exendin-4 in blood have been observed with each route of administration via the respiratory tract, and with sublingual and gavaged peptide via the gastrointestinal tract.

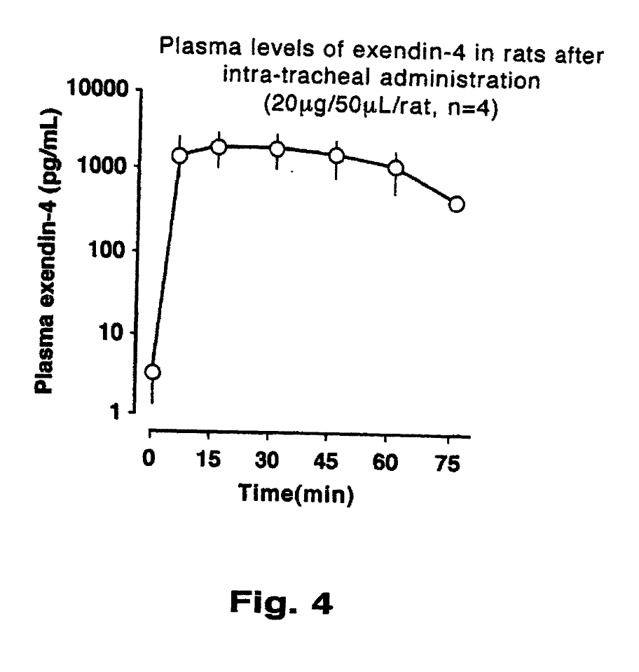

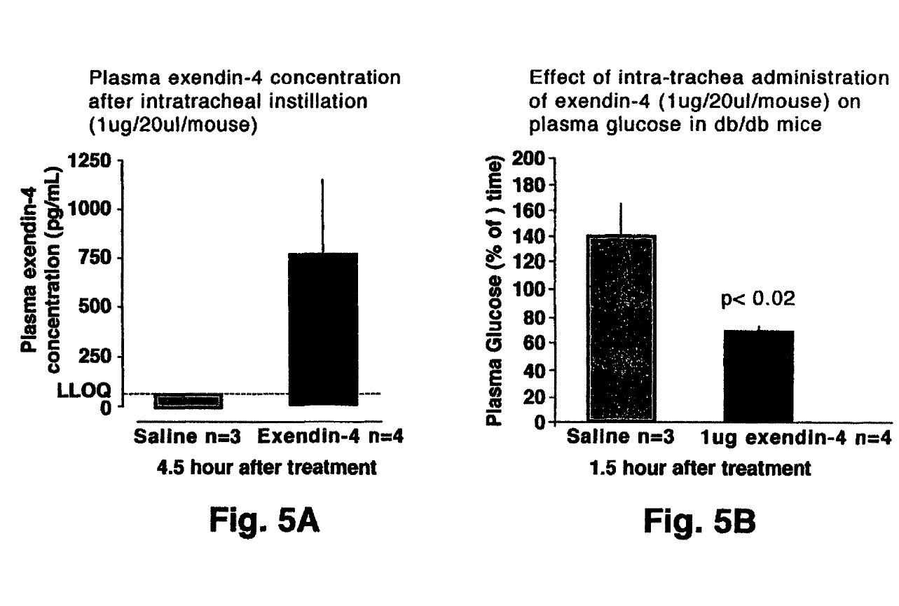

Intra-tracheal Administration—As described herein, intra-tracheal administration of exendin-4 into fasted rats (20 μg/50 μL/animal) produced a rise in the mean plasma exendin-4concentration to 2060±960 pg/mL within 5-10 minutes after administration. Elevated plasma exendin-4 concentrations were maintained for at least 1 hour after instillation (see FIG. 4). In diabetic db/db mice, intra-tracheal instillation of exendin-4 (1 μg/animal) lowered plasma glucose concentration by 30% while that in the vehicle control group increased by 41% 1.5 hours after treatment. In these animals the mean plasma concentration of exendin-4 was 777±365 pg/ml at 4.5 hours after treatment (see FIGS. 5a and 5b).

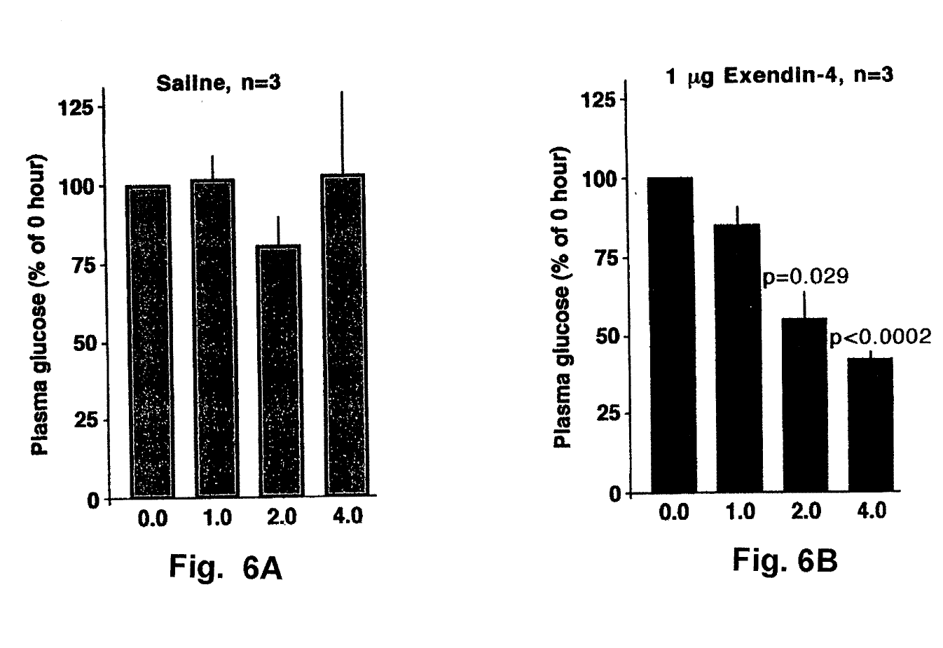

In diabetic ob/ob mice, intra-tracheal instillation of exendin-4 (1 μg/animal) decreased plasma glucose concentration to 43% of the pre-treatment level after 4 hours while that in the vehicle control group was not changed (see FIGS. 6a and 6b).

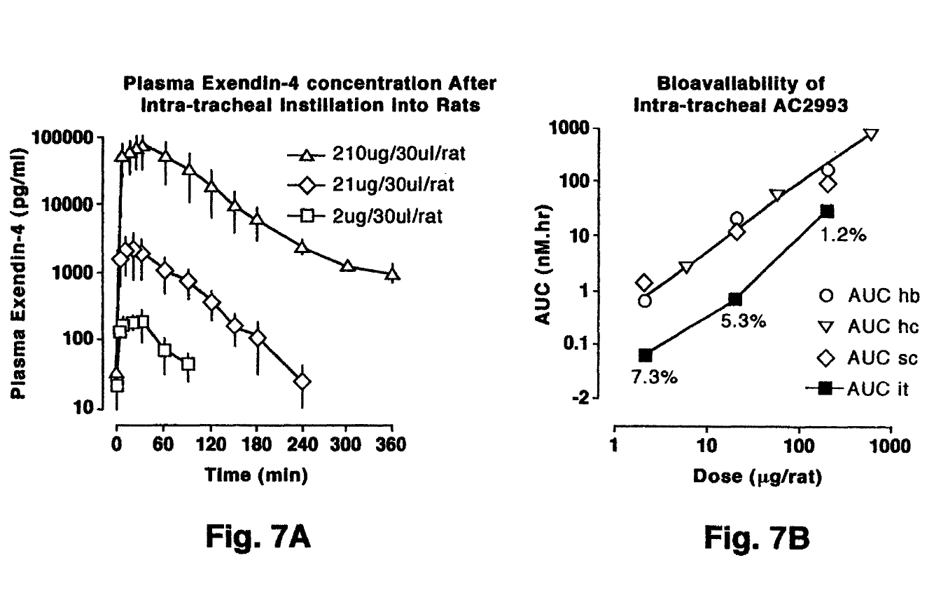

Nine overnight-fasted male Sprague Dawley rats (age 96-115 days, weight 365-395, mean 385 g) were anesthetized with halothane, tracheotomized, and catheterized via the femoral artery. At t=0 min, 30 μL of saline in which was dissolved 2.1 μg (n=3), 21 μg (n=3) or 210 μg of exendin-4 was instilled into the trachea beyond the level of intubation. Blood samples were taken after 5, 10, 20, 30, 60, 90, 120, 150, 180, 240, 300 and 360 min, centrifuged and plasma stored at −20° C. for subsequent immunoradiometric (IRMA) assay directed to N-terminal and C-terminal epitopes of the intact exendin-4 molecule. Following intra-tracheal administration, 61-74% of peak plasma concentration was observed within 5 min. Tmax occurred between 20 and 30 min after administration. AUC and Cmax were proportional to dose. At a dose of 2.1 μg (1.5 mmol/kg), resulting in plasma concentrations of ˜50 pM (where glucose-lowering effects in man are observed), bioavailability was 7.3%. The coefficient of variation was 44%. At higher doses, bioavailability was slightly lower, and the CV was higher (see FIGS. 7a and 7b). Via the tracheal route of administration, the t½ (defined pragmatically as time for plasma to fall below 50% of Cmax) was 30-60 min for the lowest dose and 60-90 min for the 2 higher doses. In sum, biologically effective quantities of exendin-4 are rapidly absorbed via the trachea without evoking apparent respiratory distress. The respiratory tract is a viable route of administration of exendin-4.

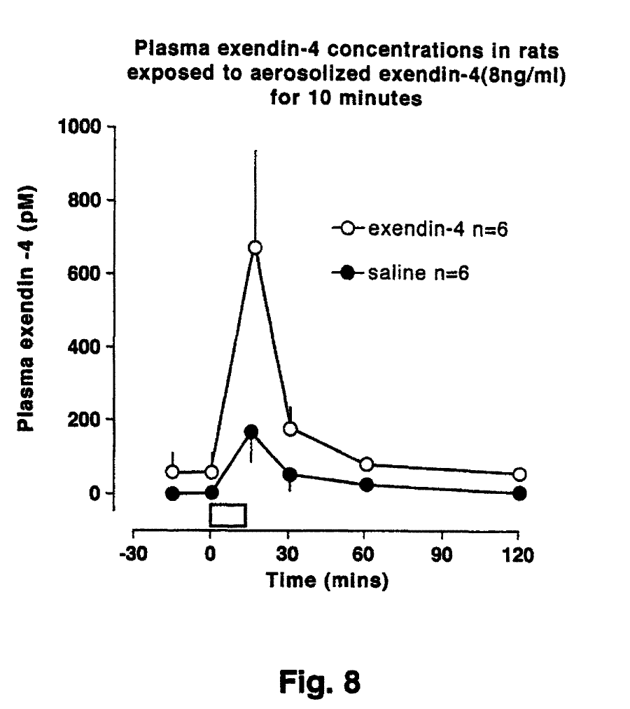

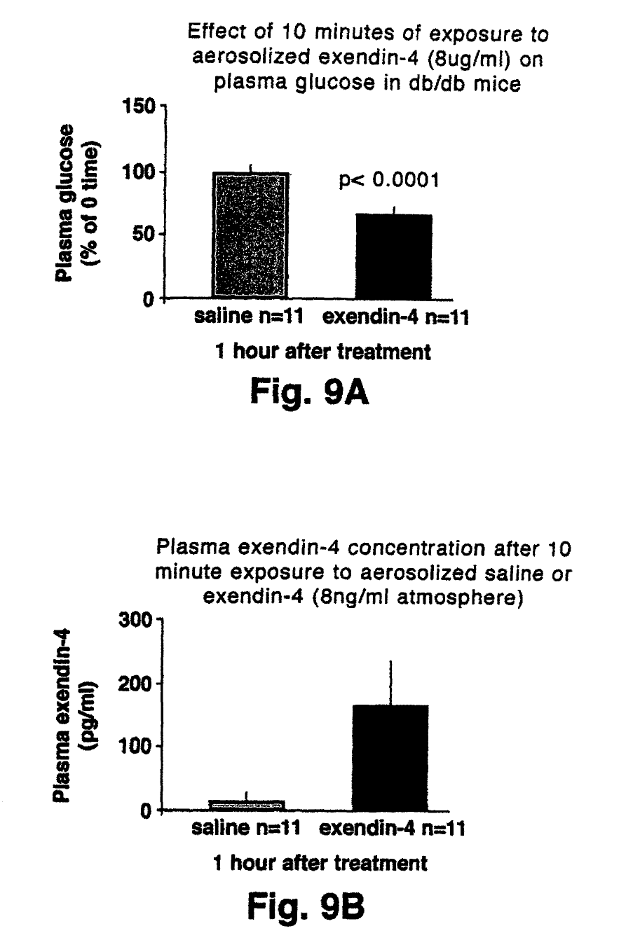

Pulmonary Administration—Increased plasma concentrations of exendin-4 were detected in rats exposed to aerosolized exendin-4. Exposure of rats to approximately 8 ng of aerosolized 10) exendin-4 per mL of atmosphere for 10 minutes resulted in peak plasma exendin-4 concentrations of 300-1900 pg/mL 5 minutes following treatment (see FIG. 8). Similar exposure of diabetic db/db mice to aerosolized exendin-4 lead to a 33% decrease in plasma glucose concentration after 1 hour, when a mean plasma exendin-4 concentration of 170±67 pg/mL was detected. Diabetic db/db mice in the control group exposed to aerosolized saline recorded no change in plasma glucose (see FIGS. 9a and 9b).

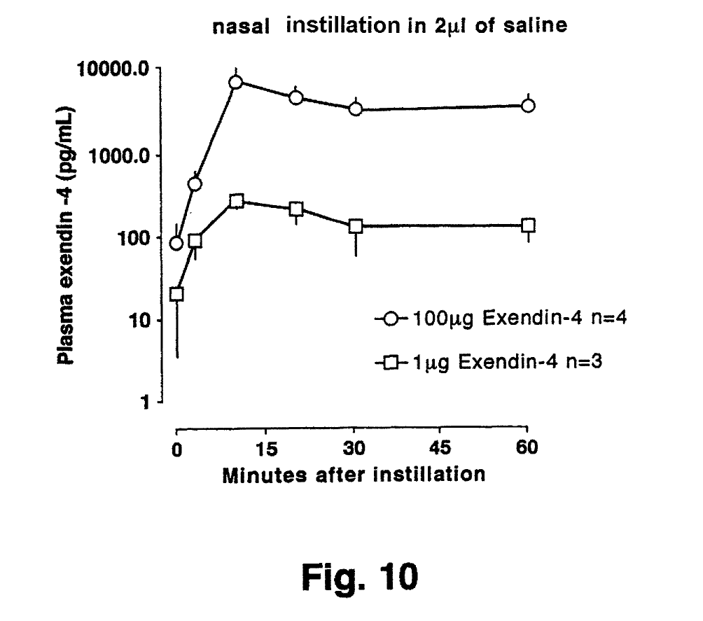

Nasal administration—Application of exendin-4 into the nasal cavity of rats led to a rise in plasma concentrations. Peak values of 300 pg/mL and 6757 pg/mL were detected 10 minutes after administration of 1 μg and 100 μg exendin-4 (dissolved in 2 μL saline), respectively (see FIG. 10).

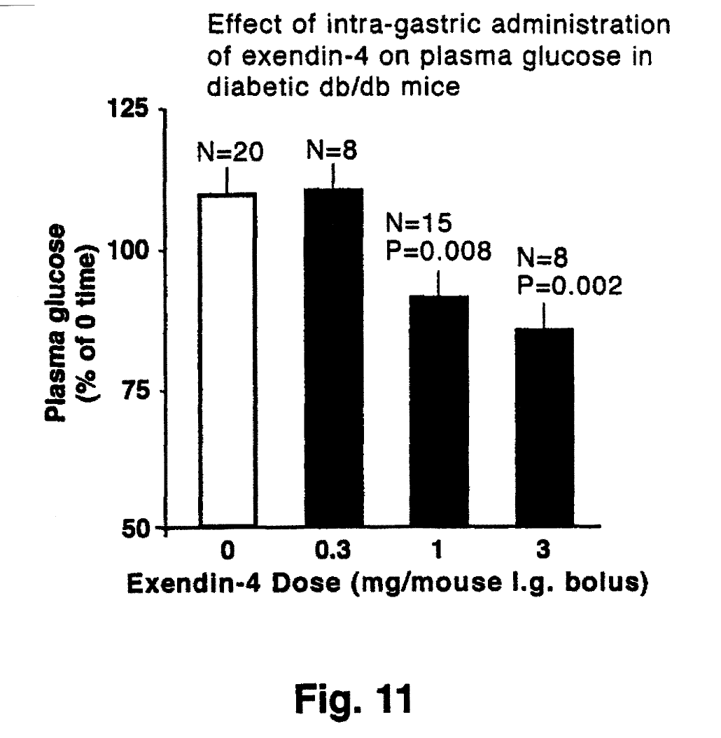

Administration via the Gut—Male db/db mice (approximately 50 g body wt.) were fasted for 2 h and before and after an intra-gastric administration of saline or exendin-4 (exendin-4). A 9% decrease in plasma glucose concentration was observed with 1 mg/200 μl/animal and a 15% decrease was observed with 3 mg/200 μl/animal, compared with a 10% increase plasma glucose in the controls one hour after treatment (see FIG. 11).

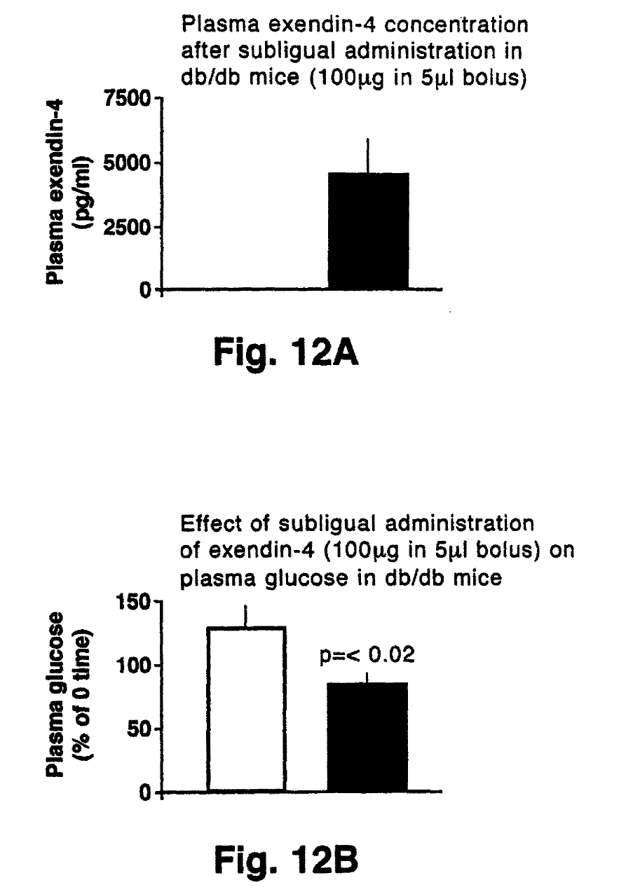

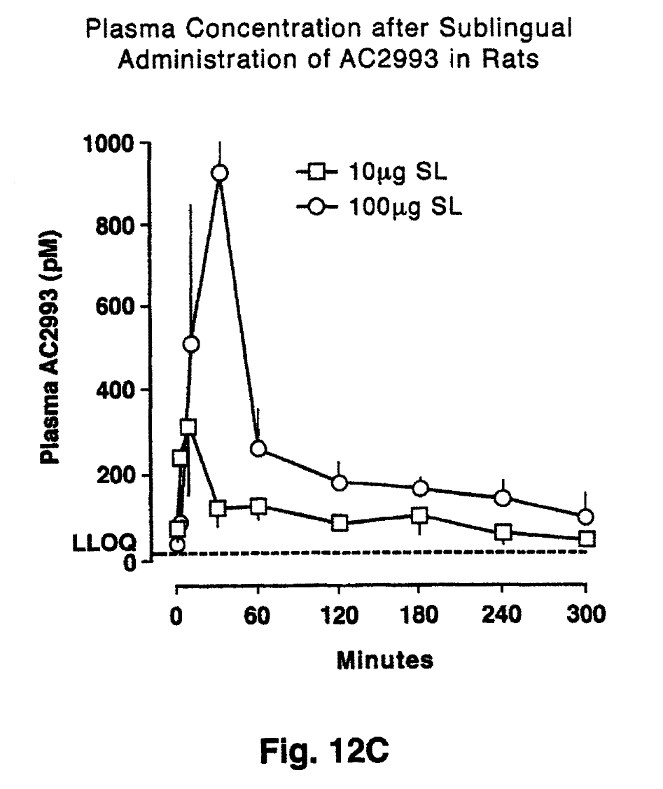

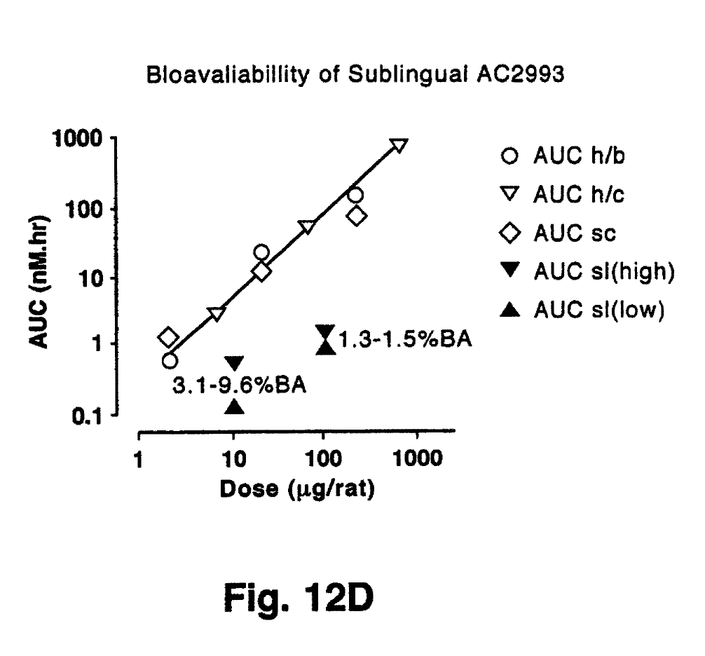

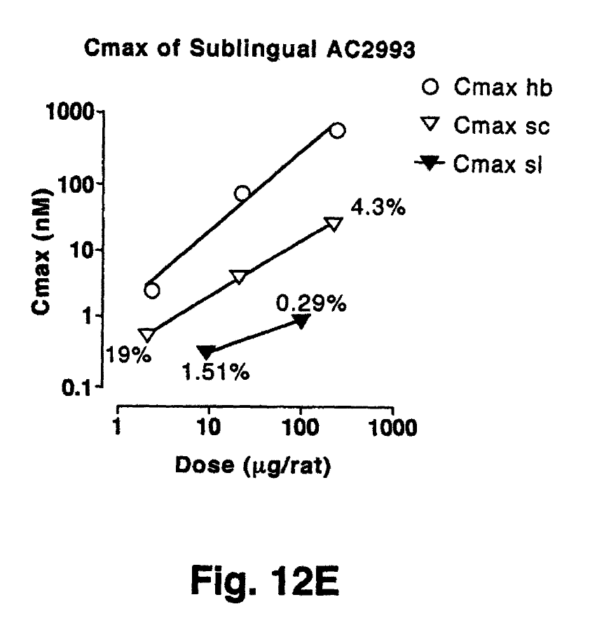

Sublingual Administration—Sublingual application of exendin-4 (100 μg/5 μL/animal) to diabetic db/db mice led to a 15% decrease in plasma glucose concentration one hour after treatment. A 30% increase was observed for the control group receiving saline. The mean exendin-4 plasma level at 60 minutes was 4520±1846 pg/mL (see FIGS. 12a, 12b, and 12c).

Eight Sprague Dawley rats (˜300 g) were briefly anesthetized with metophane while a solution containing 10 μg/3 μL (n=4) or 100 μg/3μL (n=4) was pipetted under the tongue. Blood samples were subsequently collected from the topically anesthetized tail and assayed for exendin-4 by IRMA. Plasma concentrations had begun to rise by 3 min after administration and were maximal 10 min and 30 min after administration (10 kg and 100 kg doses, respectively). Plasma exendin-4 concentration subsequently remained above the lower limit of quantitation (LLOQ) beyond 5 hours. Area-under-the-curve to the end of each experiment was calculated by the trapezoidal method. Two numbers were derived, one derived from total immunoreactivity, the other derived from the increment above the non-zero value present at t=0. These values were compared to historical intravenous bolus data in the same animal model to obtain, respectively, high and low estimates of bioavailability. For the 10 μg dose, sublingual bioavailability was 3.1-9.6%, and for a 100 μg dose, bioavailability was lower at 1.3-1.5%. Variability of AUC was greatest in the first hour after administration (CV 74% and 128% for 10 and 100 μg doses). For the 5-hour integral, coefficient of variation of the AUC was 20% and 64%, respectively. Peak plasma concentration (Cmax) occurred as rapidly after sublingual administration as after subcutaneous administration (Tmax ˜30 min). Cmax after sublingual administration of 10 μg exendin-4 was 1.5% that after an intravenous bolus, but 14.5% of that obtained after a subcutaneous bolus. Cmax after sublingual administration of 100 μg exendin-4 was only 0.29% of that observed after an intravenous bolus, and 6.1% of that obtained after a subcutaneous bolus (see FIGS. 12d and 12e). Delivery by sublingual admnistration could be enhanced by using a solid dosage form containing absorption enhancing ingredients, when placed under the tongue. Bioavailability and Cₘₐₓ were greatest, Tₘₐₓ was shortest, and variability of availability was least with the lowest sublingual dose. The lowest sublingual dose resulted in plasma concentrations similar to those that are predicted to be effective in lowering glucose in humans (˜50-100 pM).

To assist in understanding the present invention the following Examples are included which describe the results of a series of experiments. The experiments relating to this invention should not, of course, be construed as specifically limiting the invention and such variations of the invention, now known or later developed, which would be within the purview of one skilled in the art are considered to fall within the scope of the invention as described herein and hereinafter claimed.

### EXAMPLE 1

### Continuous Subcutaneous Infusion of Exendin-4 Provides Sustained Glycemic Control

This single-blind, placebo-controlled, dose-rising study was designed to compare 23-hour continuous subcutaneous infusions of four doses of exendin-4 (0.2 μg/kg/day; 0.4 μg/kg/day; 0.6 μg/kg/day; and 0.8 μg/kg/day) with placebo, in subjects with type 2 diabetes mellitus. Subjects were randomly assigned to one of five treatment sequences; within each sequence, each subject received placebo and four doses of AC2993 in a dose-rising manner. A placebo infusion was given on Day 1 and on alternate days. Subjects received a total of 10 infusions (6 placebo and 4 exendin-4) during 10 consecutive days.

A weight maintenance diet program was assigned, and subjects were given three discrete meals and an evening snack daily. Each meal and snack were consumed at the same time (±15 minutes) each day. This study further demonstrated that exendin-4 lowers plasma glucose via a number of mechanisms, among which glucose-dependent insulinotropism is prominent. This study analyzed treatment of patients with type 2 diabetes (DM2) by continuous infusion subcutaneously. Prior data have demonstrated marked effects to acutely lower post-prandial glucose and 28 day data have established the beneficial effects of improved glycemic (HbA1c) and weight control when exendin-4 is administered as a pre-meal injection twice-a-day (0.08 μg/kg). In this single-blind, placebo-controlled study, 23-hr continuous subcutaneous infusions of four doses of exendin-4 (0.2 μg/kg/day; 0.4 μg/kg/day; 0.6 μg/kg/day; 0.8 μg/kg/day) were compared with placebo in patients with DM2. Twelve patients (69-85 kg; mean (±SD) age=54±7) with DM2 inadequately controlled with metformin and/or diet (baseline HbA_{1c}: 7.4-10.6%) each received a total of 10 square wave infusions (6 placebo and 4 exendin-4) over the course of 10 consecutive days. During each infusion, plasma glucose and exendin-4 were measured at various time intervals. Serial samples of plasma were assayed using a validated immunoenzymatic assay (IEMA). This sandwich-type assay uses mouse-based monoclonal antibodies that react with exendin-4, but one or both antibodies do not react with GLP-1. The lower limit of quantitation was 2.5 pg/ml.

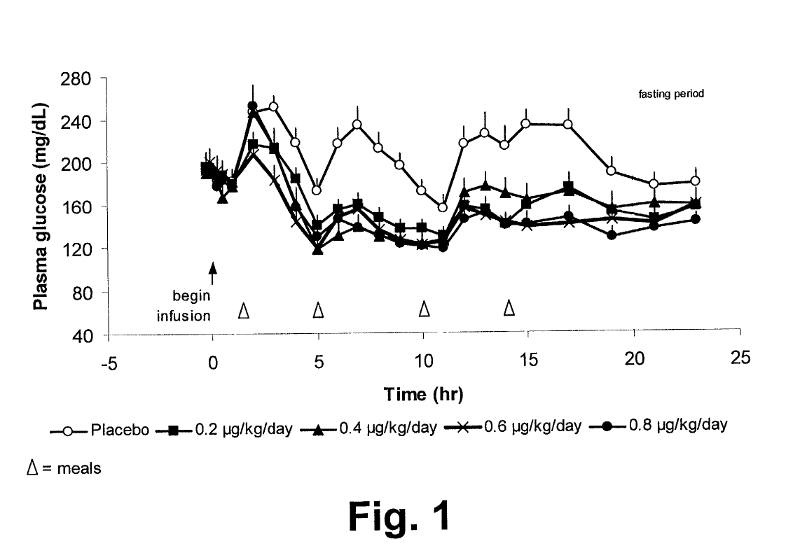

Breakfast, lunch, dinner and an evening snack were provided within the first 14 hr of the infusion. Plasma exendin-4 concentrations were dose-proportional and steady state was reached after at least 4 hr of infusion. At each time point from t=3 hr through completion of the infusion, all doses of exendin-4 lowered mean plasma glucose concentrations compared to placebo (FIG. 1).

These results demonstrate effectiveness of exendin-4 to lower glucose in preprandial, prandial, and fasting states when delivered as a subcutaneous continuous infusion in patients with DM2.

### EXAMPLE 2

### Glucose-Lowering Effects of Exendin-4 in the Fasting State

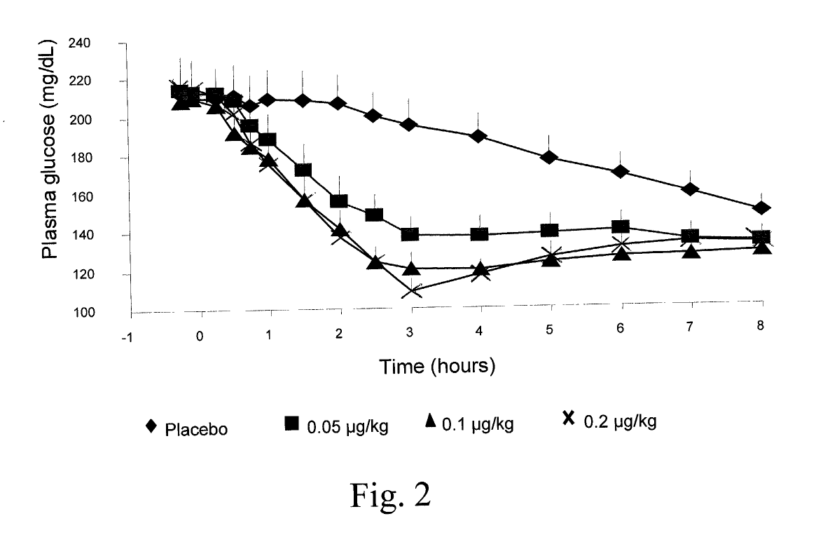

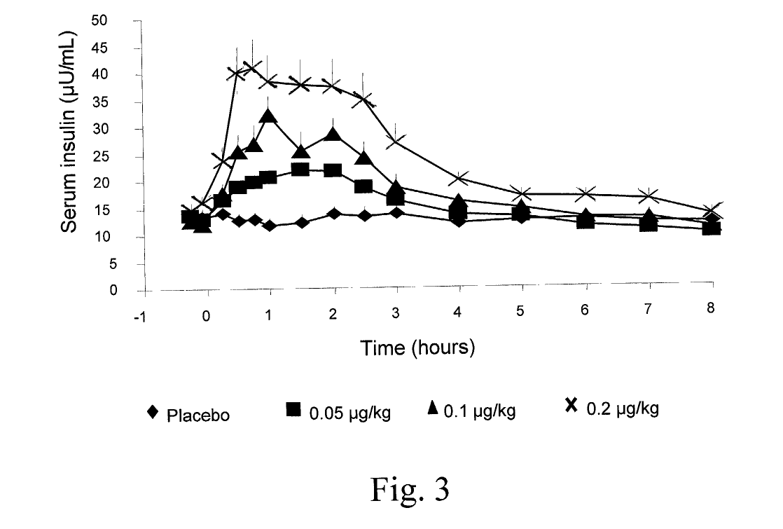

In this study, the effects of a single SC AC2993 injection on circulating glucose (FIG. 2), insulin (FIG. 3), and glucagon concentrations over 8 hours after an overnight fast were investigated. Thirteen patients with diabetes mellitus type 2 [61.5% male; (mean±SD) BMI 32.8±5.4 kg/m²; age 49±7yrs; HbA1c 9.8±1.3%; fasting plasma glucose (FPG) 221.8±41.5 mg/dL] being treated with metformin and/or thiazolidinedione were enrolled. Each patient received 3 injections of exendin-4 (0.05, 0.1, and 0.2 μg/kg) and 1 placebo (PBO) injection in random order. Mean FPG fell markedly during the 8 hour post-dose period, with FPG reaching nadir at t—3 hrs, for all exendin doses compared to PBO.

Mean serum insulin concentrations (Ins) AUC(0-8 hr) and peak Ins rose in a dose-dependent manner (FIG. 3). Ins declined rapidly near t=3 hr, coinciding with FPG nadir for all exendin doses. Incremental AUC(0-3 hr) (pg*hr/mL) for plasma glucagon concentrations were -64.3±34 (0.2 μg/kg of exendin), −63.4±42 (0.1 μg/kg), and −50.5±34 (0.05 μg/kg) compared to −22.5±26 (PBO). All doses of study medication were well tolerated. Adverse events were similar to previously reported exendin studies, consisting mainly of mild/moderate nausea; there was no hypoglycemia. We conclude that exendin effectively lowers glucose during fasting, at least in part, by glucose-dependently increasing Ins and suppressing glucagon concentrations acutely in type 2 diabetes. In addition to its potent postprandial anti-hyperglycemic effects, exendin importantly lowered FPG during the post-absorptive period. Exendin thus can provide day-long glucose control in diabetes.

### EXAMPLE 3

### Exendin-4 Decreases Glucagon Secretion During Hyperglycemic Clamps in Diabetic Fatty Zucker Rats

Absolute or relative hyperglucagonemia is often a feature of type 1 and type 2 diabetes mellitus, and the suppression of excessive glucagon secretion is a potential benefit of therapy using glucagonostatic agents. In this Example, the effect of exendin-4 on glucagon secretion in male anaesthetized Diabetic Fatty Zucker (ZDF) rats was examined. Using an hyperinsulinemic hyperglycemic clamp protocol, factors tending to influence glucagon secretion were held constant. Plasma glucose was clamped at −34 mM 60 min before beginning intravenous infusions of saline (n=7) or exendin-4 (0.21 μg+2.1 μg/mL/h; n=7). Plasma glucagon concentration measured before these infusions were similar in both groups (306±30pM versus 252±32 pM, respectively; n.s.).

Mean plasma glucagon concentration in exendin-4 infused rats was nearly half of that in saline-infused rats in the final 60 minutes of the clamp (165±18 pM versus 298±26 pM, respectively; P<0.002). The hyperglycemic clamp protocol also enabled measurement of insulin sensitivity. Glucose infusion rate during the clamp was increased by 111±17% in exendin-4-treated versus control rats (P<0.001). In other words, exendin-4 exhibited a glucagonostatic effect in ZDF rats during hyperglycemic clamp studies, an effect that will be of therapeutic benefit in diabetic humans.

### EXAMPLE 4

### Pharmacokinetics of Exendin-4 in the Rat Following Intravenous, Subcutaneous and Intraperitoneal Administration

This Example describes work to define the plasma pharmacokinetics of exendin-4 in rats (˜350 g body weight each) following 2.1, 21, 210 μg/rat i.v. bolus, s.c. and i.p. administration and 2.1, 21, 210 μg/hr/rat i.v. infusion (3 hr). Serial samples of plasma (˜120 μL) were assayed using a validated immunoradiometric assay (IRMA). This sandwich-type assay uses mouse-based monoclonal antibodies that react with exendin-4 but do not react with GLP-1 or tested metabolites of exendin-4 or GLP-1. The lower limit of quantitation was 15 pM (63 pg/mL). The estimated t_{1/2} for exendin-4 was 18-41 min for i.v. bolus, 28-49 for i.v. continuous, 90-216 min for s.c. and 125-174 min for i.p. injection. Bioavailability was 65-76% for s.c. and i.p. injection. Clearance determined from the i.v. infusion was 4-8 mL/min. Both Cₘₐₓ and AUC values within each route of administration were proportional to dose. Volume of distribution was 457-867 mL. Clearance and bioavailability were not dose dependent. Cₘₐₓ (or steady-state plasma concentration; Cₛₛ) is shown in the table below

Cmax or Css (nM)

| | Intravenous | Intravenous | | |
|---|---|---|---|---|
| Route | bolus | infusion | Subcutaneous | Intraperitoneal |
| Dose | | | | |
| 2.1 μg | 2.9 ± 0.4 | 1.1 ± 0.1 | 0.56 ± 0.12 | 0.26 ± 0.04 |
| 21 μg | 70 ± 3 | 19 ± 1.9 | 4.1 ± 1.5 | 3.9 ± 1 |
| 210 μg | 645 ± 12 | 262 ± 60 | 28 ± 4 | 35 ± 6 |

### EXAMPLE 5

### Comparison of the Insulinotropic Actions of Exendin-4 and Glucagon-Like Peptide-1 (GLP-1) During an Intravenous Glucose Challenge in Rats

This experiment compares the insulinotropic actions of synthetic exendin-4 and GLP-1 in vivo following an intravenous (i.v.) glucose challenge in rats. Sprague-Dawley rats (˜400 g) were anesthetized with halothane and cannulated via the femoral artery and saphenous vein. Following a 90-min recovery period, saline or peptide (30 pmol/kg/min each) was administered i.v. (1 ml/h for 2 hours; n=4-5 for each group). Thirty min after infusion commenced, D-glucose (5.7 mmol/kg, 0.8 ml) was injected i.v. In saline-treated, exendin-4-treated and GLP-1-treated rats, plasma glucose concentrations were similar before injection (9.3±0.3, 9.7±0.3, 10.3±0.4 mM), increased by similar amounts after glucose injection (21.7, 21.3, 23.7 mM), and resulted in a similar 60-min glucose AUC (987±39, 907±30, 1096±68 mM·min, respectively). That is, the glycemic stimulus was similar in each treatment group. Plasma insulin concentration in saline-treated rats increased 3.3-fold with the glucose challenge (230±53 to a peak of 765±188 pM). With exendin-4 infusion, the increase in plasma insulin concentration was 6.8-fold (363+60 to 2486±365 pM). With GLP-1 the increase in plasma insulin concentration was 2.9-fold (391±27 to 1145±169 pM), which was similar to that obtained in saline-treated rats. The 60-min insulin AUC in saline-treated rats was 24±6 nM·min, was increased 2.8-fold in exendin-treated rats (67±8 nM·min; P<0.003 versus saline; P<0.02 versus GLP-1) and by 20% in GLP-1-treated rats (n.s. versus saline). Amplification of glucose-stimulated insulin release by exendin-4 was also tested at infusion rates of 3 and 300 pmol/kg/min and shown to be dose-dependent.

Thus, exendin-4 is more potent and/or effective than GLP-1 in amplifying glucose-stimulated insulin release in intact rats.

### EXAMPLE 6

### Comparison of GLP-1 Receptor Binding/Activating and Glucose-Lowering Effects of GLP-1 and Exendin-4

Exendin-4 was synthesized by solid phase peptide synthesis techniques and compared to synthetic GLP-1 in terms of in vitro binding to, and activation of, GLP-1 receptors, and in vivo in terms of lowering plasma glucose in diabetic db/db mice. In a plasma membrane preparation of a rat insulinoma cell line (RINm5f) that expresses the GLP-1 receptor, the peptides were assayed for their ability to bind and displace radiolabeled GLP-1 and for their ability to stimulate the production of cAMP. The relative order of binding potency was found to be GLP-1>exendin-4. The relative order of cyclase activation was GLP-1=exendin-4. Affinities, as shown in the table below, differ over a 4- to 5-fold range. In contrast, in vivo glucose lowering potency differed over a 3430-fold range. Exendin-4 was 3430-fold more potent than GLP-1. The in vivo potency of exendin-4 does not match potency at the GLP-1 receptor, and is likely the culmination of an aggregate of properties.

| | | | Glucose-lowering |
|---|---|---|---|
| | Binding IC50 (nM) | Cyclase EC50 (nM) | ED50 (μg) |
| GLP-1 | 0.15 | 0.28 | 20.6 |
| Exendin-4 | 0.66 | 0.30 | 0.006 |

### EXAMPLE 7

### Comparison of Glycemic Indices and Insulin Sensitivity in Pair-Fed and Exendin-4-Treated Diabetic Fatty Zucker Rats

This Example tests whether the beneficial effects of exendin-4 in ZDF rats were secondary to changes in food intake. It compares effects obtained with exendin-4 to effects observed in saline-treated matched animals who consumed the same amount of food as was eaten by ZDF rats injected subcutaneously twice daily with 10 μg exendin-4. Plasma glucose and HbA1c were measured weekly for 6 weeks. One day after the last treatment, animals were anesthetized with halothane and subjected to an hyperinsulinemic (50 mU/kg/min) euglycemic clamp. Changes in HbA1c over 6 weeks differed between treatment groups (P<0.001 ANOVA), increasing in ad lib fed (n=5) and pair fed (n=5) rats, but decreasing in exendin-4-treated rats (n—5). Similarly, changes in plasma glucose differed between treatment groups (P<0.002—ANOVA), increasing in ad lib fed and pair fed ZDF rats, and decreasing in ZDF rats treated with exendin-4. In the final hour of a 3-hour clamp protocol, glucose infusion rate in exendin-4treated rats tended to be higher than in pair fed (+105%) and ad lib fed (+20%) controls, respectively (10.14±1.43 n=5, 8.46±0.87 n=4, 4.93±2.02 mg/kg/min n=3; n.s. P=0.09 ANOVA). Another index of insulin sensitivity, plasma lactate concentration, differed significantly between treatment groups (P<0.02 ANOVA) and was lowest in exendin-4-treated rats. Thus, exendin-4 treatment is associated with improvement in glycemic indices and in insulin sensitivity that is partly, but not fully, matched in controls fed the same amount of food, indicating that improvements in metabolic control with exendin-4 in ZDF rats are at least partly due to mechanisms beyond caloric restriction.

### EXAMPLE 8

### Clinical Studies and the Stimulation of Endogenous Insulin Secretion by Subcutaneous Synthetic Exendin-4 in Healthy Overnight Fasted Volunteers

In a double blind, placebo-controlled single ascending dose clinical trial to explore safety and tolerability and pharmacokinetics of synthetic exendin-4, exendin-4 formulated for subcutaneous injection was evaluated in healthy male volunteers while assessing effects upon plasma glucose and insulin concentrations. Five single subcutaneous doses of exendin-4 (0.01, 0.05, 0.1, 0.2 or 0.3 μg/kg) were studied in 40 healthy male volunteers in the fasting state. Maximum plasma exendin-4 concentrations were achieved between 1 and 2 hours post-dose with little difference among the doses examined. Examination of the data indicated a dose dependent increase for Cₘₐₓ. There were no serious adverse events reported in this study and in the healthy male volunteers that participated in this study, exendin-4 was well tolerated at subcutaneous doses up to and including 0.1 μg/kg. A decrease in plasma glucose concentration was also observed at this dose. At doses of 0.2 μg/kg and higher, the most commonly observed adverse events were headache, nausea, vomiting, dizziness, and postural hypotension. There was a transient fall in plasma glucose concentration following administration of doses of 0.05 μg/kg and above.

Forty healthy, lean (mean BMI (±SE) 22.7±1.2) subjects aged 18-40 years were randomly assigned to 5 groups. Within each group of 8 subjects, 6 were assigned to exendin-4 and 2 to placebo (PBO). Exendin-4 (0.01, 0.05, 0.1, 0.2 or 0.3 μg/kg) or placebo was administered following an overnight fast and plasma exendin-4, glucose and insulin concentrations monitored along with safety and tolerability. No safety issues were observed. Doses ≦0.1 μg/kg were tolerated as well as PBO whereas 0.2 and 0.3 μg/kg elicited a dose-dependent increase in nausea and vomiting. Peak plasma exendin-4 concentrations rose dose-dependently and following 0.1 μg/kg, exendin-4 immunoreactivity persisted for 360 min. Plasma glucose decreased following all doses, except 0.01 μg/kg, reached a nadir by 30 min and returned back to baseline within 180 min. Subjects receiving 0.3 μg/kg received a caloric beverage 30 minutes after dosing, precluding comparison of their data. Mean change in plasma glucose (0-180 min): 0.03±0.07, −0.07±0.08, −0.38±0.14, −0.85±0.13 and −0.83±0.23 mmol/L for PBO, 0.01, 0.05, 0.1, and 0.2 μg/kg respectively; P≦0.02 versus PBO. The lowest plasma glucose recorded was 3.4 mmol/L. Corresponding mean changes in plasma insulin (0-120 min) were 0.43±0.59, 2.37±0.58, 2.28±0.66, 4.91±1.23, and 14.00±3.34 μU/mL; P≦0.01 versus PBO for the 0.1 and 0.2 μg/kg groups. Thus, in healthy, overnight fasted volunteers, subcutaneous injection of exendin-4 (1) presented no safety issues, (2) was well-tolerated at doses ≦0.1 μg/kg, (3) led to exendin-4 immunoreactivity in plasma for up to 6 hrs, (4) increased plasma insulin and lowered plasma glucose in a dose-dependent manner without inducing hypoglycemia.

### EXAMPLE 9

### Effectiveness of Alternate Delivery of Exendin-4 in Rodents

This Example tested the delivery of exendin-4 by means alternative to injection, and examined its ability to traverse mucosal surfaces in sufficient quantities to exert biological effect. Changes in concentration of plasma glucose and of intact synthetic exendin-4 (measured by a 2-site immunoradiometric assay) were observed in db/db mice administered a saline solution containing differing doses of synthetic exendin-4 via the trachea, via an aerosol mist (pulmonary), via gavage (oral), and under the tongue (sublingual).

For tracheal administration, male db/db mice (approximately 50 g) were fasted for 2 hours, and the trachea was intubated under anesthesia. The animals were bled (75 μl, orbital sinus) before and after 20 μl saline or 1 μg exendin-4 dissolved in saline was administered into the trachea of each animal. Plasma exendin and glucose levels were determined (FIGS. 5a and 5b).

For intra-gastric administration, male db/db mice (˜50 g each) were fasted for 2 hours and bled (40 μl, orbital sinus) before and one hour after 200 μl saline was administered in a bolus dose (0, 0.3, 1, and 3 mg/mouse) intra-gastrically into each animal (effects on plasma glucose per dose, FIG. 11).

Sublingual application application of exendin (100 μg/animal in 5 μl) to diabetic db/db mice led to a 15% decrease in plasma glucose concentration one hour after treatment. A 30% increase was observed for the control group receiving saline. The mean exendin plasma level at 60 min was 4520±1846 pg/ml. FIGS. 12A and 12B.

The same routes of administration, as well as intraduodenally and nasally, were tested in rats, and bioavailability was calculated, for example, for sublingual and intra-tracheal routes. Male rats (350-400 g) fasted overnight were cannulated in the trachea and femoral artery under anesthesia. Blood was drawn from the arterial lime before and after (5, 15, 30, 45, 60, and 75 min) 20 μg of exendin-4 dissolved in 50 μl saline was administered into the trachea of each rat. Plasma exendin levels were determined with an immunoradiometric assay (FIG. 4).

For pulmonary administration, male rats (approximately 350 grams each) fasted overnight were placed in a two liter chamber and exposed to aerosolized exendin-4 for 10 min. .O Exendin-4 was nebulized at a rate of 0.2 mg/min at a flow rate of 5 L/min. The concentration of aerosolized exendin-4 was extimated from samples of chamber atmosphere drawn during the course of the experiment. Results are shown in FIG. 8. Similar exposure in db/db mice produced effects on glucose and exendin plasma levels as shown in FIGS. 9A and 9B.

For nasal instillation, Harnal Sprague Dawley rats (311-365 g each), nonfasted, were dosed with 0, 1, or 100 μg of exendin-4 in 2 μl of saline by application into the nostrils. Blood samples from anesthetized (Hurricane) tail tips were collected at 0, 3, 10, 20, 30, and 60 min after dosing, and exendin plasma levels were measured by IRMA (FIG. 10).

Exendin-4 administered via each of the above routes in mice resulted in significant glucose-lowering activity 1 to 4 hours after administration (db/db mice intra-tracheal P<0.02; ob/ob mice intra-tracheal P<0.0002; db/db mice aerosol P<0.0001; gavage P<0.002; sublingual P<0.02). Dose-dependent increases in plasma exendin-4 concentration were up to 777±365 pg/mL (db/db mice intra-tracheal); 170±67 pg/mL (db/db mice aerosol); 4520+1846 pg/mL (db/db mice sublingual; FIGS. 12A and 12B). Similarly, in rats, exendin-4 concentrations were observed up to 68,682±38,661 pg/mL (intra-tracheal; FIG. 4); 1900 pg/mL (pulmonary); 6757 pg/mL (nasal); 3,862±2,844 pg/mL (sublingual; FIGS. 12C, 12D, 12E); but no apparent absorption or biological activity when delivered intraduodenally. Bioavailability of exendin-4 in saline was 7.3% at lower doses when delivered via the trachea, where 61-74% of Cmax was observed within 5 min. Kinetics thereafter were similar to those observed after subcutaneous administration. Bioavailability of exendin-4 in saline delivered under the tongue was 3.1-9.6% at lower doses. These studies support the delivery of exendin-4 and peptide agonist analogs thereof in biologically effective quantities via convenient non-injectable routes.

### EXAMPLE 10

### A Single-Blind, Placebo Controlled Study on the Metabolic Effects of a Range of Doses of Synthetic Exendin-4 Given by Subcutaneous Injection to People with Type 2 Diabetes Mellitus

This Example describes the results of a two-part, single-blind, placebo controlled study to examine the metabolic effects of a range of doses of synthetic exendin-4 given by the 1) subcutaneous route to subjects with Type II diabetes mellitus. The subjects involved in the study were individuals diagnosed with Type II diabetes and being controlled with diet and/or with oral hypoglycemic agents (OHAs) and with HbA_{1c} concentration ≧7.0% but ≦12.0% at the screening visit.

The study commenced with a screening visit, after which the subjects taking OHAs were instructed to stop this medication and return to the clinic approximately 14 days later when the effects of the OHA dissipated. Subjects who participated in Part 1 arrived at the clinic the afternoon prior to the first dose and began the three or four scheduled dosing days. Each dosing event was scheduled to be 24 hours apart.

Following consent and screening, subjects were randomly assigned to receive synthetic exendin-4 or placebo. In the first portion of the study, six subjects were confined to an in-patient clinical research unit for three to four days and assigned to one of 4 treatment sequences, where they were to receive each of the following doses: placebo or synthetic exendin-4 at 0.1 or 0.01, or possibly 0.001 μg/kg. Doses were administered subcutaneously following an overnight fast. A standardize liquid meal was given 15 minutes after injection of the study medication. The table below illustrates the dosing schedule for Part 1:

| | Day 1 | Day 2 | Day 3 | Day 4* |
|---|---|---|---|---|
| Subject 1 | Placebo | 0.1 μg/kg | 0.01 μg/kg | 0.001 μg/kg |
| Subject 2 | Placebo | 0.1 μg/kg | 0.01 μg/kg | 0.001 μg/kg |
| Subject 3 | 0.1 μg/kg | Placebo | 0.01 μg/kg | 0.001 μg/kg |
| Subject 4 | 0.1 μg/kg | Placebo | 0.01 μg/kg | 0.001 μg/kg |
| Subject 5 | 0.1 μg/kg | 0.01 μg/kg | Placebo | 0.001 μg/kg |
| Subject 6 | 0.1 μg/kg | 0.01 μg/kg | Placebo | 0.001 μg/kg |

*only be completed if an effect on glucose was observed on Day 3.

In the second part of the study, approximately three days after the completion of Part 1, eight subjects were also confined to an in-patient clinical research unit for four days. The subjects were different subjects from those who participated in Part 1. The study procedures and schedule of events during Part 2 were consistent with Part 1. The doses were determined after the effect on glucose in Part 1 was analyzed.

Because there was no significant effect seen at 0.01 μg/kg during Part 1, subjects were dosed according to the following schedule in Part 2:

| | Day 1 | Day 2 | Day 3 | Day 4 |
|---|---|---|---|---|
| Group A | Placebo | 0.02 μg/kg | 0.05 μg/kg | 0.1 μg/kg |
| Group B | 0.02 μg/kg | 0.1 μg/kg | Placebo | 0.05 μg/kg |
| Group C | 0.05 μg/kg | Placebo | 0.1 μg/kg | 0.02 μg/kg |
| Group D | 0.1 μg/kg | 0.05 μg/kg | 0.02 μg/kg | Placebo |

Subjects who participated in Part 2 began their dosing following review of the data from Part 1 in the same manner. All subjects returned to the clinic 4 to 6 days after discharge from the in-patient unit for a safety reassessment.

The synthetic exendin-4 used for the study was a clear colorless sterile solution for subcutaneous injection, formulated in sodium acetate buffer (pH 4.5) and containing 4.3% mannitol as an iso-osmolality modifier. The strength of synthetic exendin-4 injection was 0.1 mg/mL. One mL of solution was supplied in 3 mL vials with rubber stoppers. Placebo solution was made from the same sterile formulation but without the drug substance, synthetic exendin-4.

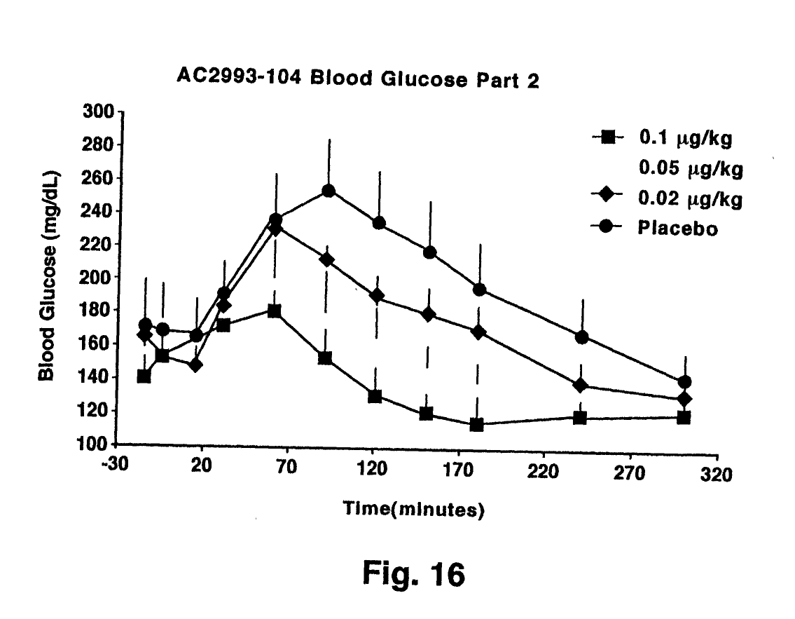

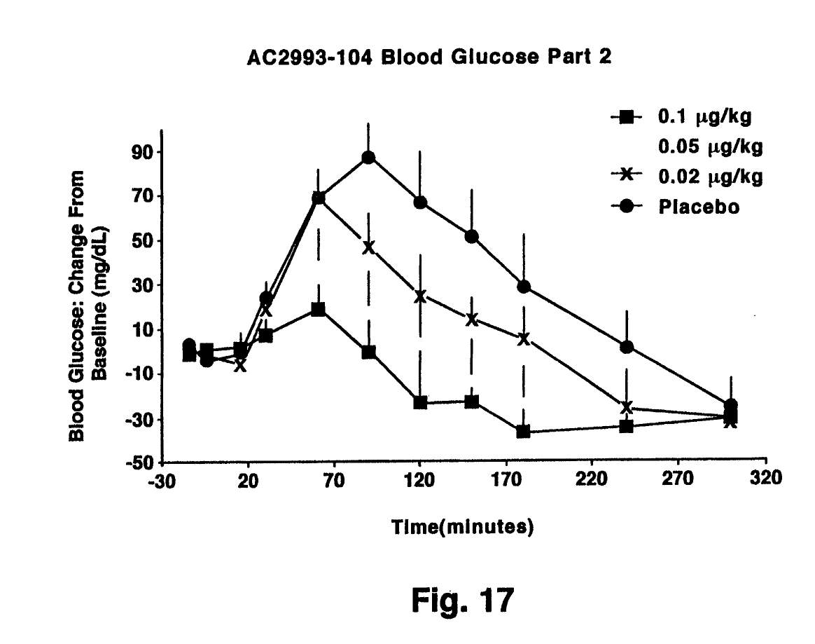

The results of the study are shown in FIGS. 16 and 17. They indicate the ability of various different doses of exendin-4 (0.02 μg/kg, 0.05 μg/kg, and 0.1 μg/kg) to lower blood glucose in people with Type 2 diabetes.

### EXAMPLE 11

This Example describes an experiment to determine a dose-response for the insulin-sensitizing effects of exendin-4 and agonists thereof in Diabetic Fatty Zucker rats. The exendin-4 used in these studies was obtained from Bachem (Torrance, Calif.; Cat H8730, Lot 506189), American Peptides (Sunnyvale, Calif.; Cat 301577, Lot K10051TI) and from in-house solid-phase synthesis (lot AR1374-11; peptide content 93.3%). Thirty nine male Diabetic fatty Zucker rats 0.1E0 (ZDF)/Gmi™-(fa/fa) (age 116±20 days; weight 441±39 g) were assigned to 5 treatment groups: saline injections only (n=9), exendin-4 injections 0.1, 1, 10 or 100 μg (n=9, 10, 6, 5, respectively). Of these, 35 rats were used in hyperinsulinemic euglycemic clamp studies (n=9, 7, 9, 5, 5, respectively). Blood was sampled from the tip of the topically-anesthetized tail (Hurricaine brand of 20% topical benzocaine solution, Beutlich, Waukegan, Ill.) of conscious overnight-fasted rats before treatment and at weekly intervals for 5 weeks during treatment for analysis of hemoglobin A_{1c} (DCA2000 latex immuno-agglutination inhibition, Bayer Diagnostics, Tarrytown, N.Y.). Body weight was measured daily.

After 6 weeks of treatment, ˜16 hours after the last exendin-4 (or saline) dose, and after an overnight fast, hyperinsulinemic euglycemic clamps (DeFronzo R A, Tobin J D, Andres R: Glucose clamp technique: a method for quantifying insulin secretion and resistance. Amer J Physiol 237:E214-23 ,1979) were performed on halothane-anesthetized rats. Rats were thermoregulated, tracheotomized and catheterized via the saphenous vein for infusion of 20% D-glucose and insulin, and via the femoral artery for blood sampling and blood pressure monitoring (P23XL transducer, Spectramed, Oxnard, Calif.; universal amplifier, Gould, Valley View, Ohio; A/D conversion, DataTranslation, Wilmington, Del.). Insulin (Humulin-R, Eli Lilly, Indianapolis, Ind.) was infused at 50 mU/kg/min, beginning at t-30 min and continued until t=+180 min. Glucose was infused at a variable rate to maintain euglycemia, determined by glucose sampling and analysis at 5 min intervals (immobilized glucose oxidase method; YSI 2300-Stat Analyzer, Yellow Springs, Ohio). Mean plasma glucose during clamps was 103.9 mg/dL (mean coefficient of variation was 5.8%). Glucose infusion rate data for analysis were taken from t—60-180 min when responses had approached a steady state. Plasma lactate data, obtained from an immobilized lactate oxidase sensor incorporated in the glucose analyzer, were also collected.

Injections were given intraperitoneally at ˜8 a.m. and 4 p.m., Monday through Friday, and at ˜10 a.m. on Saturday and Sunday.

Pairwise statistical analyses were performed using Student's t-test routines (Instat v3.0, GraphPad Software, San Diego, Calif.) using P<0.05 as the level of significance. Dose-response -1 analyses used 4-parameter logistic regression and general effects were tested using one-way ANOVA (Prism v3.0, GraphPad Software, San Diego, Calif.).

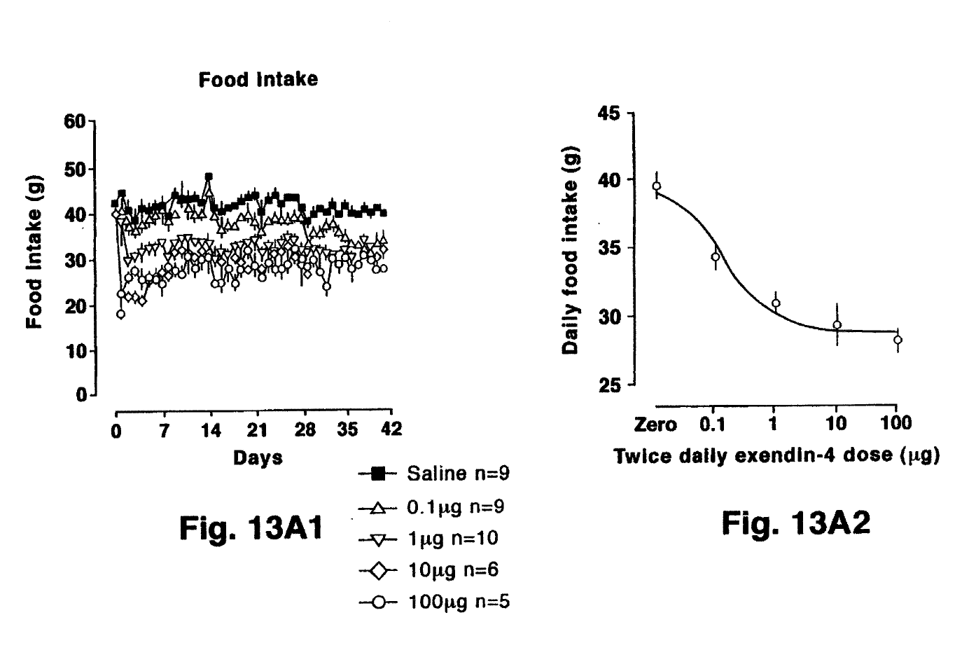

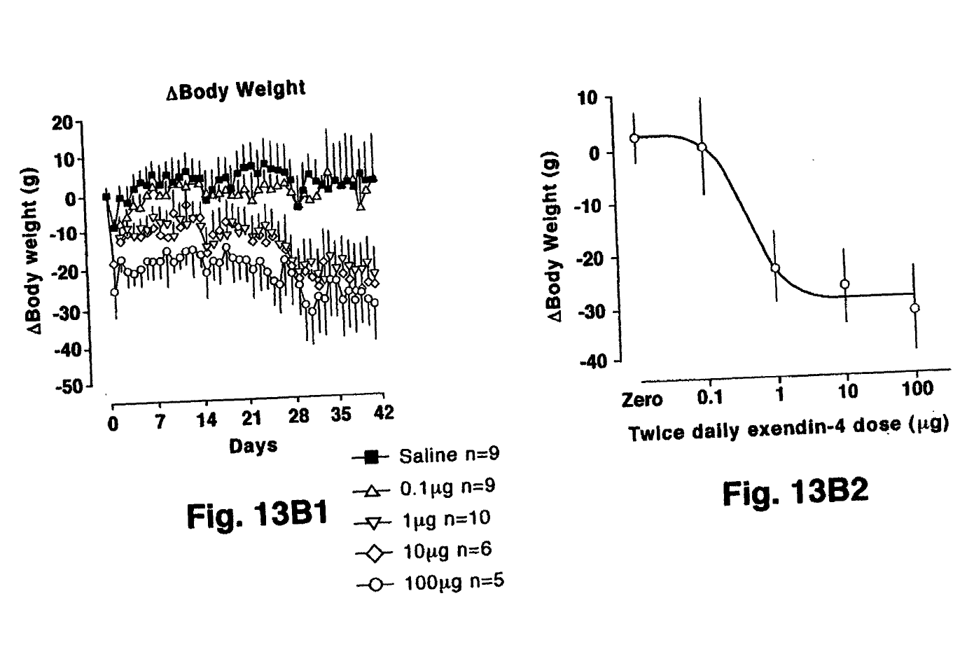

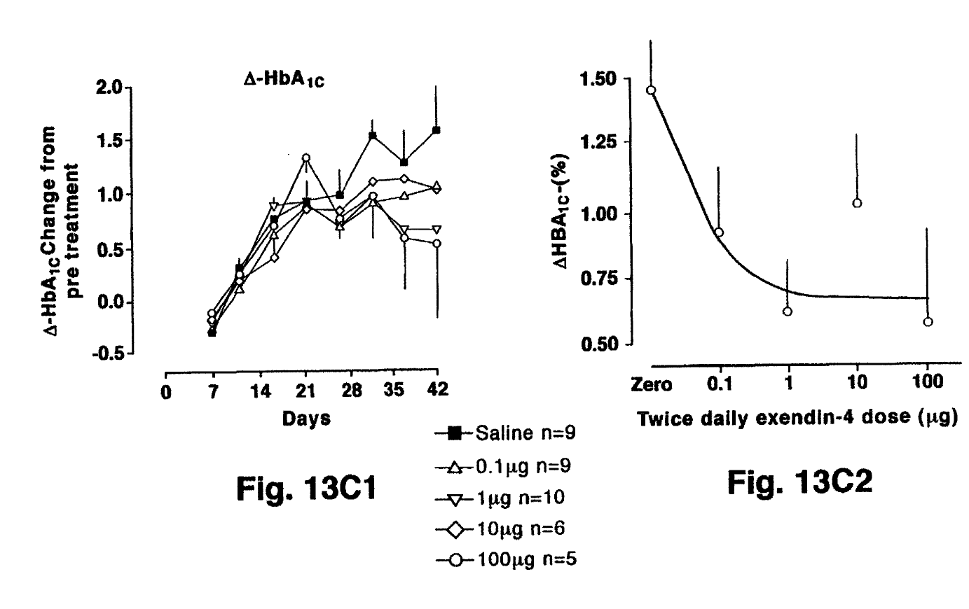

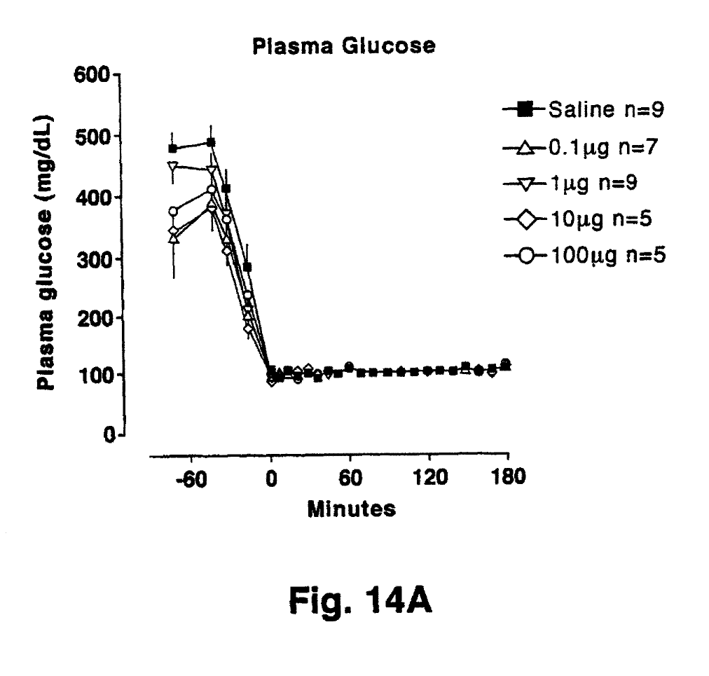

The results showed that in Diabetic Fatty Zucker rats treated with different doses of exendin-4 for 6 weeks, there was a dose-dependent reduction in food intake (ED50 0.14 μg±0.15 log; see FIG. 13a), and in body weight (ED50 0.42 μg±0.15 log; see FIG. 13b) of up to 27±2 g, representing a 5.6±0.5% decrease in body weight relative to saline-injected controls.

In this group of rats, the diabetic course appeared progressive, since hemoglobin A_{1c} initially rose in all groups. Injection of exendin-4 nonetheless appeared to dose-dependently arrest and reverse the rise in hemoglobin A_{1c} (see FIG. 13c). The exendin-4 dose-response for effect on hemoglobin A1c measured during the last 2 weeks of treatment was generally significant (P=0.05 ANOVA) and specifically at 1 μg and 100 μg doses (P<0.005, P<0.02 respectively). A similar pattern was observed in relation to fasting plasma triglycerides in the last 2 weeks of treatment, where plasma concentrations were significantly reduced at all doses by between 51% and 65% (P<0.002 ANOVA).

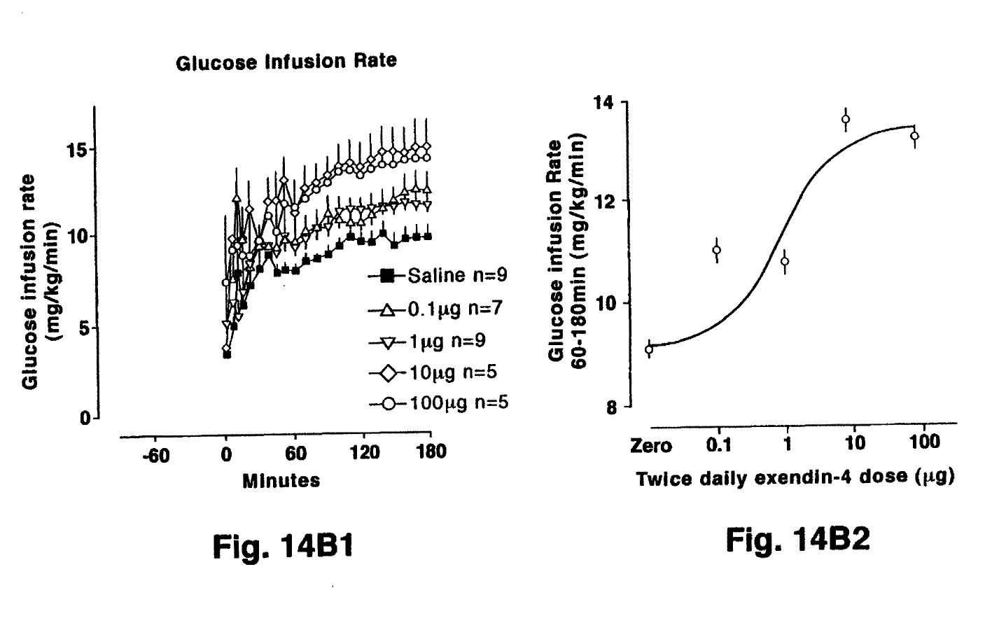

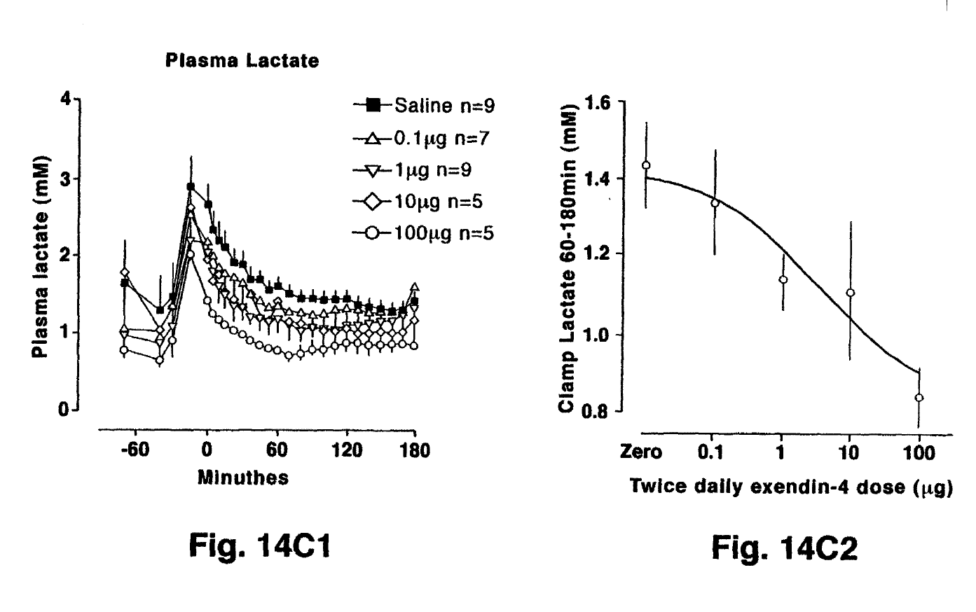

Thirty five of the 39 rats entered into the study progressed to an hyperinsulinemic, euglycemic clamp ˜16 hours after their last treatment. Initial fasting plasma glucose concentrations, higher in saline-treated (489±28 mg/dL) than exendin-treated rats, fell with insulin infusion and were subsequently clamped at similar plasma glucose concentrations (105.6 mg/dL at 60-180 min; mean coefficient of variation 4.6%; see FIG. 14a). Glucose infusion rate required to maintain euglycemia was dose-dependently increased by prior treatment with exendin-4 (ED50 1.0 μg±0.41 log; see FIG. 14b). Exendin-4 treatment increased glucose infusion rate by up to 48% relative to saline-treated controls.

Plasma lactate concentration before and during the clamp procedure was dose-dependently reduced by prior treatment with exendin-4 (ED50 4 μg±0.25 log; see FIG. 14c). This effect, representing up to a 42% reduction in mean plasma lactate concentration between 60 and 180 minutes of the clamp, appeared primarily due to a reduction in pre-clamp (basal) lactate concentration; increments in plasma lactate during hyperinsulinemia were similar in all treatment groups. There were no treatment-related differences in mean arterial pressure measured before or during clamp procedures.

The approximately 50% increase in insulin sensitivity observed after chronic administration of exendin-4 was both important and surprising in view of observations that exendin-4 has no acute effect in insulin-sensitive tissues in vitro (i.e. no effect on basal or insulin-stimulated incorporation of radiolabeled glucose into glycogen in isolated soleus muscle, or into lipid in isolated adipocytes; Pittner et al., unpublished). Although the possibility that the increase in insulin sensitivity may have resulted in some part from improved glycemic control and reduced glucose toxicity may not be overlooked, it has been reported that the increase in insulin sensitivity from various antidiabetic therapies, including those not classed as insulin sensitizing, is quite variable and it has been reported that acute treatment with GLP-1 appears not to immediately alter insulin sensitivity in humans (Orskov L, Holst J J, Moller J, Orskov C, Moller N, Alberti K G, Schmitz O: GLP-1 does not not acutely affect insulin sensitivity in healthy man. Diabetologia 39:1227-32, 1996; Ahren B, Larsson H, Holst J J: Effects of glucagon-like peptide-1 on islet function and insulin sensitivity in noninsulin-dependent diabetes mellitus. J Clin Endocrinol Metab 82:473-8, 1997; UK Prospective Diabetes Study Group: Intensive blood-glucose control with sulphonylureas or insulin compared with conventional treatment and risk of complications in patients with type 2 diabetes (UKPDS 33). Lancet 352:837-53, 1998). Thus chronic administration of exendin-4 appears to be associated with increases in insulin sensitivity that are as great as, if not greater than, those observed with other therapies, including insulin sensitizing drugs such as thiazolidinediones and metformin.

### EXAMPLES A TO E

Reagents Used

GLP-1 [7-36]NH₂ (GLP-1) was purchased from Bachem (Torrance, Calif.). All other peptides were prepared using synthesis methods such as those described therein. All chemicals were of the highest commercial grade. The cAMP SPA immunoassay was purchased from Amersham. The radioligands were purchased from New England Nuclear (Boston, Mass.). RINm5f cells (American Type Tissue Collection, Rockville, Md.) were grown in DME/F12 medium containing 10% fetal bovine serum and 2 mM L-glutamine. Cells were grown at 37° C. and 5% CO₂/95% humidified air and medium was replaced every 2 to 3 days. Cells were grown to confluence then harvested and homogenized using on a Polytron homogenizer. Cell homogenates were stored frozen at −70° C. until used.

### EXAMPLE A

### GLP-1 Receptor Binding Studies

Receptor binding was assessed by measuring displacement of [¹²⁵I]GLP-1 or [¹²⁵I]exendin(9-39) from RINm5f membranes. Assay buffer contained 5 μg/ml bestatin, 1 μg/ml phosphoramidon, 1 mg/ml bovine serum albumin (fraction V), 1 mg/ml bacitracin, and 1 mM MgCl₂ in 20 mM HEPES, pH 7.4. To measure binding, 30 μg membrane protein (Bradford protein assay) was resuspended in 200 μl assay buffer and incubated with 60 pM [¹²⁵I]GLP-1 or [¹²⁵I]exendin(9-39) and unlabeled peptides for 120 minutes at 23□C in 96 well plates (Nagle Nunc, Rochester, N.Y.). Incubations were terminated by rapid filtration with cold phosphate buffered saline, pH 7.4, through polyethyleneimine-treated GF/B glass fiber filters (Wallac Inc., Gaithersburg, Md.) using a Tomtec Mach II plate harvester (Wallac Inc., Gaithersburg, Md.). Filters were dried, combined with scintillant, and radioactivity determined in a Betaplate liquid scintillant counter (Wallac Inc.).

Peptide samples were run in the assay as duplicate points at 6 dilutions over a concentration range of 10⁻⁶M to 10⁻¹²M to generate response curves. The biological activity of a sample is expressed as an IC₅₀ value, calculated from the raw data using an iterative curve-fitting program using a 4-parameter logistic equation (Prizm, GraphPAD Software).

### EXAMPLE B

### Cyclase Activation Study

Assay buffer contained 10 μM GTP, 0.75 mM ATP, 2.5 mM MgCl₂, 0.5 mM phosphocreatine, 12.5 U/ml creatine kinase, 0.4 mg/ml aprotinin, 1 μM IBMX in 50 mM HEPES, pH 7.4. Membranes and peptides were combined in 100 ml of assay buffer in 96 well filter-bottom plates (Millipore Corp., Bedford, Mass.). After 20 minutes incubation at 37° C., the assay was terminated by transfer of supernatant by filtration into a fresh 96 well plate using a Millipore vacuum manifold. Supernatant cAMP contents were quantitated by SPA immunoassay. Peptide samples were run in the assay as triplicate points at 7 dilutions over a concentration range of 10⁻⁶M to 10⁻¹²M to generate response curves. The biological activity of a particular sample was expressed as an EC₅₀ value, calculated as described above.

### EXAMPLE C

### Determination of Blood Glucose Levels in db/db Mice

C57BLKS/J-m-db mice at least 3 months of age were utilized for the study. The mice were obtained from The Jackson Laboratory and allowed to acclimate for at least one week -before use. Mice were housed in groups often at 22° C.±1° C. with a 12:12 light:dark cycle, with lights on at 6 a.m. All animals were deprived of food for 2 hours before taking baseline blood samples. Approximately 70 μl of blood was drawn from each mouse via eye puncture, after a light anesthesia with metophane. After collecting baseline blood samples, to measure plasma glucose concentrations, all animals receive subcutaneous injections of either vehicle (10.9% NaCl), exendin-4 or test compound (1 μg) in vehicle. Blood samples were drawn again, using the same procedure, after exactly one hour from the injections, and plasma glucose concentrations were measured. For each animal, the % change in plasma value, from baseline value, was calculated.

### EXAMPLE D

### Dose Response Determination of Blood Glucose Levels in db/db Mice

C57BLKS/J-m-db/db mice, at least 3 months of age were utilized for the study. The mice were obtained from The Jackson Laboratory and allowed to acclimate for at least one week before use. Mice were housed in groups often at 22° C.±1° C. with a 12:12 light:dark cycle, with lights on at 6 a.m. All animals were deprived of food for 2 hours before taking baseline blood samples. Approximately 70 μl of blood was drawn from each mouse via eye puncture, after a light anesthesia with metophane. After collecting baseline blood samples, to measure plasma glucose concentrations, all animals receive subcutaneous injections of either vehicle, exendin-4 or test compound in concentrations indicated. Blood samples were drawn again, using the same procedure, after exactly one hour from the injections, and plasma glucose concentrations were measured. For each animal, the % change in plasma value, from baseline value, was calculated and a dose dependent relationship was evaluated using Graphpad Prizm™ software.

### EXAMPLE E

### Gastric Emptying

The following study was and may be carried out to examine the effects of exendin-4 and/or an exendin agonist compound on gastric emptying in rats. This experiment followed a modification of the method of Scarpignato, et al., Arch. Int. Pharmacodyn. Ther. 246:286-94, 1980. Male Harlan Sprague Dawley (HSD) rats were used. All animals were housed at 22.7±0.8 C in a 12:12 hour light:dark cycle (experiments being performed during the light cycle) and were fed and watered ad libitum (Diet LM-485, Teklad, Madison, Wis.). Exendin-4 was synthesized according to standard peptide synthesis methods. The preparation of exendin-4 is described in Example 14. The determination of gastric emptying by the method described below was performed after a fast of ˜20 hours to ensure that the stomach contained no chyme that would interfere with spectrophotometric absorbance measurements.

Conscious rats received by gavage, 1.5 ml of an acaloric gel containing 1.5% methyl cellulose (M-0262, Sigma Chemical Co, St Louis, Mo.) and 0.05% phenol red indicator. Twenty minutes after gavage, rats were anesthetized using 5% halothane, the stomach exposed and clamped at the pyloric and lower esophageal sphincters using artery forceps, removed and opened into an alkaline solution which was made up to a fixed volume. Stomach content was derived from the intensity of the phenol red in the alkaline solution, measured by absorbance at a wavelength of 560 nm. In separate experiments on 7 rats, the stomach and small intestine were both excised and opened into an alkaline solution. The quantity of phenol red that could be recovered from the upper gastrointestinal tract within 20 minutes of gavage was 89±4%; dye which appeared to bind irrecoverably to the gut luminal surface may have accounted for the balance. To account for a maximal dye recovery of less than 100%, percent of stomach contents remaining after 20 min were expressed as a fraction of the gastric contents recovered from control rats sacrificed immediately after gavage in the same experiment. Percent gastric contents remaining=(absorbance at 20 min)/(absorbance at 0 mm)×100.

Various modifications of the invention in addition to those shown and described herein will become apparent to those skilled in the art from the foregoing description and fall within the scope of the following claims.

We claim:

1. A pharmaceutical composition comprising an exendin or an exendin agonist peptide in an extended release formulation, the formulation being capable of releasing the peptide over a predetermined release period, the period being at least one hour, in an amount such that, when the composition is administered to a human, an average sustained plasma level of at least 5 pg/ml is achieved for at least 25% of the predetermined release period.

2. The composition of claim 1 wherein the plasma level is achieved for at least 50% of the predetermined release period.

3. The composition of claim 1 wherein the plasma level is achieved for at least 75% of the predetermined release period.

4. The composition of claim 1 wherein the plasma level is at least 40 pg/ml.

5. The composition of claim 2 wherein the plasma level is at least 40 pg/ml.

6. The composition of claim 3 wherein the plasma level is at least 40 pg/ml.

7. The composition of claim 1 wherein the plasma level is at least 1100 pg/ml.

8. The composition of claim 2 wherein the plasma level is at least 100 pg/ml.

9. The composition of claim 3 wherein the plasma level is at least 100 pg/ml.

10. A method of administering an exendin or an exendin agonist to a patient in need thereof, comprising administering the exendin or agonist to the patient in an amount from about 0.0005 μg/kg/dose to about 12000 μg/kg/dose.

11. The method of claim 10 wherein the exendin or agonist is administered parenterally in an amount from about 0.001 μg/kg/dose to about 1.0 μg/kg/dose.

12. The method of claim 11 wherein the parenteral administration is subcutaneous.

13. The method of claim 11 wherein the parenteral administration is via an implanted or injected slow release composition designed to prolong administration over the time course of at least one hour.

14. The method of claim 13 wherein the slow release composition is designed to prolong administration over at least one day.

15. The method of claim 13 wherein the slow release composition is designed to prolong administration over at least one week.

16. The method of claim 13 wherein the slow release composition is designed to prolong administration over at least one month.

17. The method of claim 10 wherein the exendin or agonist is administered via a nasal, oral, buccal, sublingual, intra-tracheal, trans-dermal, trans-mucosal, or pulmonary route.

18. The method of claim 10 further comprising providing the exendin or agonist over a time period of at least one hour such that an average sustained plasma level of at least 5 pg/ml is achieved for at least 50% of the administered time period.

19. The method of claim 10 further comprising providing the exendin or agonist over a time period of at least one hour such that an average sustained plasma level of at least 40 pg/ml is achieved for at least 75% of the administered time period.

20. The method of claim 10 further comprising providing the exendin or agonist over a time period of at least one hour such that an average sustained plasma level of at least 40 pg/ml is achieved for at least 90% of the administered time period.

21. The method of claim 10 further comprising providing the exendin or agonist over a time period of at least 24 hours such that an average sustained plasma level of at least 5 pg/ml is achieved for at least 50% of the administered time period.

22. The method of claim 10 further comprising providing the exendin or agonist over a time period of at least one week such that an average sustained plasma level of at least 5 pg/ml is achieved for at least 50% of the administered time period.

23. The method of claim 10 further comprising providing the exendin or agonist over a time period of at least one month such that an average sustained plasma level of at least 5 pg/ml is achieved for at least 50% of the administered time period.

24. The method of claim 21 wherein the average sustained plasma level is at least 40 pg/ml.

25. The method of claim 22 wherein the average sustained plasma level is at least 40 pg/ml.

26. The method of claim 23 wherein the average sustained plasma level is at least 40 pg/ml.

27. The method of claim 10 wherein the exendin or agonist is administered in single or divided doses over the time course of one day.

28. The method of claim 10 wherein the patient has diabetes mellitus.

29. The method of claim 10 wherein the patient has impaired glucose tolerance.

30. The method of claim 10 wherein the patient is obese.

31. The method of claim 10 wherein the patient is hyperglycemic.

32. The method of claim 10 wherein the patient has dyslipidemia.

33. The method of claim 10 wherein the patient has cardiovascular disease.

34. The method of claim 18 wherein the average sustained plasma levels of the exendin or agonist do not exceed about 500 pg/ml.

35. The method of claim 18 wherein the average sustained plasma levels of the exendin or agonist do not exceed about 200 pg/ml.

36. The method of claim 18 wherein the average sustained plasma levels of the exendin or agonist do not exceed about 100 pg/ml.

37. The method of claim 18 wherein the average sustained plasma levels of the exendin or agonist do not exceed about 60 pg/ml.

38. The method of claim 10 wherein the exendin or agonist is administered to the patient from one to four times per day.

39. The method of claim 10 wherein the exendin or agonist is administered to the patient two times per day.

40. The pharmaceutical composition of claim 1 wherein the exendin agonist is an exendin analogue.


SEQUENCE LISTING (188 sequences)

SEQ ID NO: 1 - protein, 39 aa, Heloderma horridum
  c-term amidation
HSDGTFTSDLSKQMEEEAVRLFIEWLKNGGPSSGAPPPS

SEQ ID NO: 2 - protein, 39 aa, Heloderma suspectum
  c-term amidation
HGEGTFTSDLSKQMEEEAVRLFIEWLKNGGPSSGAPPPS

SEQ ID NO: 3 - protein, 39 aa, Artificial Sequence
  Description of Artificial Sequence Exendin Agonist
XXXGTXXXXXSKQXEEEAVRLXXXXLKNGGXSSGAXXXX

SEQ ID NO: 4 - protein, 38 aa, Artificial Sequence
  Description of Artificial Sequence Exendin Agonist
XXXGXXXXXXXXXXXXXAXXXXXXXXXXGGXSSGAXXX

SEQ ID NO: 5 - protein, 39 aa, Artificial Sequence
  Description of Artificial Sequence Exendin Agonist
XXXXXXXXXXXXXXXXXAXXXXXXXXXXGGXSSGAXXXX

SEQ ID NO: 6 - protein, 30 aa, Artificial Sequence
  Description of Artificial Sequence Exendin Agonist
HGEGTFTSDLSKQMEEEAVRLFIEWLKNGG

SEQ ID NO: 7 - protein, 30 aa, Artificial Sequence; identical to SEQ ID NO: 6
  Description of Artificial Sequence Exendin Agonist

SEQ ID NO: 8 - protein, 28 aa, Artificial Sequence
  Description of Artificial Sequence Exendin Agonist
HGEGTFTSDLSKQLEEEAVRLAIEFLKN

SEQ ID NO: 9 - protein, 39 aa, Artificial Sequence
  Description of Artificial Sequence Exendin Agonist
HGEGTFTSDLSKQLEEEAVRLFIEFLKNGGPSSGAPPPS

SEQ ID NO: 10 - protein, 39 aa, Artificial Sequence
  Description of Artificial Sequence Exendin Agonist
HGEGTFTSDLSKQLEEEAVRLFIEWLKNGGPSSGAPPPS

SEQ ID NO: 11 - protein, 39 aa, Artificial Sequence
  Description of Artificial Sequence Exendin Agonist
HGEGTFTSDLSKQMEEEAVRLFIEFLKNGGPSSGAPPPS

SEQ ID NO: 12 - protein, 39 aa, Artificial Sequence
  Description of Artificial Sequence Exendin Agonist
YGEGTFTSDLSKQMEEEAVRLFIEWLKNGGPSSGAPPPS

SEQ ID NO: 13 - protein, 39 aa, Artificial Sequence
  Description of Artificial Sequence Exendin Agonist
HGEGTFTSDLSKQMEEEAVRLFIEWLKNGGPSSGAPPPY

SEQ ID NO: 14 - protein, 39 aa, Artificial Sequence
  Description of Artificial Sequence Exendin Agonist
HGDGTFTSDLSKQMEEEAVRLFIEWLKNGGPSSGAPPPS

SEQ ID NO: 15 - protein, 39 aa, Artificial Sequence
  Description of Artificial Sequence Exendin Agonist
HGEGTXTSDLSKQMEEEAVRLFIEWLKNGGPSSGAPPPS

SEQ ID NO: 16 - protein, 39 aa, Artificial Sequence
  Description of Artificial Sequence Exendin Agonist
HGEGTFSSDLSKQMEEEAVRLFIEWLKNGGPSSGAPPPS

SEQ ID NO: 17 - protein, 39 aa, Artificial Sequence
  Description of Artificial Sequence Exendin Agonist
HGEGTFSTDLSKQMEEEAVRLFIEWLKNGGPSSGAPPPS

SEQ ID NO: 18 - protein, 39 aa, Artificial Sequence
  Description of Artificial Sequence Exendin Agonist
HGEGTFTTDLSKQMEEEAVRLFIEWLKNGGPSSGAPPPS

SEQ ID NO: 19 - protein, 39 aa, Artificial Sequence
  Description of Artificial Sequence Exendin Agonist
HGEGTFTSELSKQMEEEAVRLFIEWLKNGGPSSGAPPPS

SEQ ID NO: 20 - protein, 39 aa, Artificial Sequence
  Description of Artificial Sequence Exendin Agonist
HGEGTFTSDXSKQMEEEAVRLFIEWLKNGGPSSGAPPPS

SEQ ID NO: 21 - protein, 39 aa, Artificial Sequence
  Description of Artificial Sequence Exendin Agonist
HGEGTFTSDXSKQLEEEAVRLFIEFLKNGGPSSGAPPPS

SEQ ID NO: 22 - protein, 39 aa, Artificial Sequence
  Description of Artificial Sequence Exendin Agonist
HGEGTFTSDLSKQXEEEAVRLFIEWLKNGGPSSGAPPPS

SEQ ID NO: 23 - protein, 39 aa, Artificial Sequence
  Description of Artificial Sequence Exendin Agonist
HGEGTFTSDLSKQXEEEAVRLFIEFLKNGGPSSGAPPPS

SEQ ID NO: 24 - protein, 39 aa, Artificial Sequence
  Description of Artificial Sequence Exendin Agonist
HGEGTFTSDLSKQMEEEAVRLXIEWLKNGGPSSGAPPPS

SEQ ID NO: 25 - protein, 39 aa, Artificial Sequence
  Description of Artificial Sequence Exendin Agonist
HGEGTFTSDLSKQMEEEAVRLFVEWLKNGGPSSGAPPPS

SEQ ID NO: 26 - protein, 39 aa, Artificial Sequence
  Description of Artificial Sequence Exendin Agonist
HGEGTFTSDLSKQLEEEAVRLFVEFLKNGGPSSGAPPPS

SEQ ID NO: 27 - protein, 39 aa, Artificial Sequence
  Description of Artificial Sequence Exendin Agonist
HGEGTFTSDLSKQMEEEAVRLFXEWLKNGGPSSGAPPPS

SEQ ID NO: 28 - protein, 39 aa, Artificial Sequence
  Description of Artificial Sequence Exendin Agonist
HGEGTFTSDLSKQLEEEAVRLFXEFLKNGGPSSGAPPPS

SEQ ID NO: 29 - protein, 39 aa, Artificial Sequence
  Description of Artificial Sequence Exendin Agonist
HGEGTFTSDLSKQMEEEAVRLFIDWLKNGGPSSGAPPPS

SEQ ID NO: 30 - protein, 39 aa, Artificial Sequence
  Description of Artificial Sequence Exendin Agonist
HAEGTFTSDLSKQLEEEAVRLFIEFLKNGGPSSGAPPPS

SEQ ID NO: 31 - protein, 39 aa, Artificial Sequence
  Description of Artificial Sequence Exendin Agonist
HGEGTFTSDLSKQMEEEAVRLFIEWLKNGGXSSGAXXXS

SEQ ID NO: 32 - protein, 39 aa, Artificial Sequence
  Description of Artificial Sequence Exendin Agonist
HGEGTFTSDLSKQMEEEAVRLFIEWLKNGGPSSGAXXXS

SEQ ID NO: 33 - protein, 39 aa, Artificial Sequence; identical to SEQ ID NO: 31
  Description of Artificial Sequence Exendin Agonist

SEQ ID NO: 34 - protein, 39 aa, Artificial Sequence; identical to SEQ ID NO: 32
  Description of Artificial Sequence Exendin Agonist

SEQ ID NO: 35 - protein, 39 aa, Artificial Sequence
  Description of Artificial Sequence Exendin Agonist
HGEGTFTSDLSKQLEEEAVRLFIEFLKNGGXSSGAXXXS

SEQ ID NO: 36 - protein, 39 aa, Artificial Sequence; identical to SEQ ID NO: 35
  Description of Artificial Sequence Exendin Agonist

SEQ ID NO: 37 - protein, 39 aa, Artificial Sequence; identical to SEQ ID NO: 31
  Description of Artificial Sequence Exendin Agonist

SEQ ID NO: 38 - protein, 39 aa, Artificial Sequence; identical to SEQ ID NO: 32
  Description of Artificial Sequence Exendin Agonist

SEQ ID NO: 39 - protein, 39 aa, Artificial Sequence; identical to SEQ ID NO: 35
  Description of Artificial Sequence Exendin Agonist

SEQ ID NO: 40 - protein, 28 aa, Artificial Sequence
  Description of Artificial Sequence Exendin Agonist
HGEGTFTSDLSKQMEEEAVRLFIEWLKN

SEQ ID NO: 41 - protein, 28 aa, Artificial Sequence
  Description of Artificial Sequence Exendin Agonist
HGEGTFTSDLSKQLEEEAVRLFIEFLKN

SEQ ID NO: 42 - protein, 28 aa, Artificial Sequence
  Description of Artificial Sequence Exendin Agonist
HAEGTFTSDLSKQLEEEAVRLFIEFLKN

SEQ ID NO: 43 - protein, 28 aa, Artificial Sequence
  Description of Artificial Sequence Exendin Agonist
HGEGAFTSDLSKQLEEEAVRLFIEFLKN

SEQ ID NO: 44 - protein, 28 aa, Artificial Sequence
  Description of Artificial Sequence Exendin Agonist
HGEGTATSDLSKQLEEEAVRLFIEFLKN

SEQ ID NO: 45 - protein, 28 aa, Artificial Sequence
  Description of Artificial Sequence Exendin Agonist
HGEGTFTADLSKQLEEEAVRLFIEFLKN

SEQ ID NO: 46 - protein, 28 aa, Artificial Sequence
  Description of Artificial Sequence Exendin Agonist
HGEGTFTSDASKQLEEEAVRLFIEFLKN

SEQ ID NO: 47 - protein, 28 aa, Artificial Sequence
  Description of Artificial Sequence Exendin Agonist
HGEGTFTSDLAKQLEEEAVRLFIEFLKN

SEQ ID NO: 48 - protein, 28 aa, Artificial Sequence
  Description of Artificial Sequence Exendin Agonist
HGEGTFTSDLSAQLEEEAVRLFIEFLKN

SEQ ID NO: 49 - protein, 28 aa, Artificial Sequence
  Description of Artificial Sequence Exendin Agonist
HGEGTFTSDLSKALEEEAVRLFIEFLKN

SEQ ID NO: 50 - protein, 28 aa, Artificial Sequence
  Description of Artificial Sequence Exendin Agonist
HGEGTFTSDLSKQAEEEAVRLFIEFLKN

SEQ ID NO: 51 - protein, 28 aa, Artificial Sequence
  Description of Artificial Sequence Exendin Agonist
HGEGTFTSDLSKQLAEEAVRLFIEFLKN

SEQ ID NO: 52 - protein, 28 aa, Artificial Sequence
  Description of Artificial Sequence Exendin Agonist
HGEGTFTSDLSKQLEAEAVRLFIEFLKN

SEQ ID NO: 53 - protein, 28 aa, Artificial Sequence
  Description of Artificial Sequence Exendin Agonist
HGEGTFTSDLSKQLEEAAVRLFIEFLKN

SEQ ID NO: 54 - protein, 28 aa, Artificial Sequence
  Description of Artificial Sequence Exendin Agonist
HGEGTFTSDLSKQLEEEAARLFIEFLKN

SEQ ID NO: 55 - protein, 28 aa, Artificial Sequence
  Description of Artificial Sequence Exendin Agonist
HGEGTFTSDLSKQLEEEAVALFIEFLKN

SEQ ID NO: 56 - protein, 28 aa, Artificial Sequence
  Description of Artificial Sequence Exendin Agonist
HGEGTFTSDLSKQLEEEAVRAFIEFLKN

SEQ ID NO: 57 - protein, 28 aa, Artificial Sequence
  Description of Artificial Sequence Exendin Agonist
HGEGTFTSDLSKQLEEEAVRLFIAFLKN

SEQ ID NO: 58 - protein, 28 aa, Artificial Sequence
  Description of Artificial Sequence Exendin Agonist
HGEGTFTSDLSKQLEEEAVRLFIEALKN

SEQ ID NO: 59 - protein, 28 aa, Artificial Sequence
  Description of Artificial Sequence Exendin Agonist
HGEGTFTSDLSKQLEEEAVRLFIEFAKN

SEQ ID NO: 60 - protein, 28 aa, Artificial Sequence
  Description of Artificial Sequence Exendin Agonist
HGEGTFTSDLSKQLEEEAVRLFIEFLAN

SEQ ID NO: 61 - protein, 28 aa, Artificial Sequence
  Description of Artificial Sequence Exendin Agonist
HGEGTFTSDLSKQLEEEAVRLFIEFLKA

SEQ ID NO: 62 - protein, 38 aa, Artificial Sequence
  Description of Artificial Sequence Exendin Agonist
HGEGTFTSDLSKQMEEEAVRLFIEWLKNGGPSSGAPPP

SEQ ID NO: 63 - protein, 38 aa, Artificial Sequence
  Description of Artificial Sequence Exendin Agonist
HGEGTFTSDLSKQLEEEAVRLFIEFLKNGGPSSGAPPP

SEQ ID NO: 64 - protein, 37 aa, Artificial Sequence
  Description of Artificial Sequence Exendin Agonist
HGEGTFTSDLSKQMEEEAVRLFIEWLKNGGPSSGAPP

SEQ ID NO: 65 - protein, 37 aa, Artificial Sequence
  Description of Artificial Sequence Exendin Agonist
HGEGTFTSDLSKQLEEEAVRLFIEFLKNGGPSSGAPP

SEQ ID NO: 66 - protein, 36 aa, Artificial Sequence
  Description of Artificial Sequence Exendin Agonist
HGEGTFTSDLSKQMEEEAVRLFIEWLKNGGPSSGAP

SEQ ID NO: 67 - protein, 36 aa, Artificial Sequence
  Description of Artificial Sequence Exendin Agonist
HGEGTFTSDLSKQLEEEAVRLFIEFLKNGGPSSGAP

SEQ ID NO: 68 - protein, 35 aa, Artificial Sequence
  Description of Artificial Sequence Exendin Agonist
HGEGTFTSDLSKQMEEEAVRLFIEWLKNGGPSSGA

SEQ ID NO: 69 - protein, 35 aa, Artificial Sequence
  Description of Artificial Sequence Exendin Agonist
HGEGTFTSDLSKQLEEEAVRLFIEFLKNGGPSSGA

SEQ ID NO: 70 - protein, 34 aa, Artificial Sequence
  Description of Artificial Sequence Exendin Agonist
HGEGTFTSDLSKQMEEEAVRLFIEWLKNGGPSSG

SEQ ID NO: 71 - protein, 34 aa, Artificial Sequence
  Description of Artificial Sequence Exendin Agonist
HGEGTFTSDLSKQLEEEAVRLFIEFLKNGGPSSG

SEQ ID NO: 72 - protein, 33 aa, Artificial Sequence
  Description of Artificial Sequence Exendin Agonist
HGEGTFTSDLSKQMEEEAVRLFIEWLKNGGPSS

SEQ ID NO: 73 - protein, 33 aa, Artificial Sequence
  Description of Artificial Sequence Exendin Agonist
HGEGTFTSDLSKQLEEEAVRLFIEFLKNGGPSS

SEQ ID NO: 74 - protein, 32 aa, Artificial Sequence
  Description of Artificial Sequence Exendin Agonist
HGEGTFTSDLSKQMEEEAVRLFIEWLKNGGPS

SEQ ID NO: 75 - protein, 32 aa, Artificial Sequence
  Description of Artificial Sequence Exendin Agonist
HGEGTFTSDLSKQLEEEAVRLFIEFLKNGGPS

SEQ ID NO: 76 - protein, 31 aa, Artificial Sequence
  Description of Artificial Sequence Exendin Agonist
HGEGTFTSDLSKQMEEEAVRLFIEWLKNGGP

SEQ ID NO: 77 - protein, 31 aa, Artificial Sequence
  Description of Artificial Sequence Exendin Agonist
HGEGTFTSDLSKQLEEEAVRLFIEFLKNGGP

SEQ ID NO: 78 - protein, 30 aa, Artificial Sequence
  Description of Artificial Sequence Exendin Agonist
HGEGTFTSDLSKQLEEEAVRLFIEFLKNGG

SEQ ID NO: 79 - protein, 29 aa, Artificial Sequence
  Description of Artificial Sequence Exendin Agonist
HGEGTFTSDLSKQMEEEAVRLFIEWLKNG

SEQ ID NO: 80 - protein, 29 aa, Artificial Sequence
  Description of Artificial Sequence Exendin Agonist
HGEGTFTSDLSKQLEEEAVRLFIEFLKNG

SEQ ID NO: 81 - protein, 38 aa, Artificial Sequence
  Description of Artificial Sequence Exendin Agonist
HGEGTFTSDLSKQMEEEAVRLFIEWLKNGGXSSGAXXX

SEQ ID NO: 82 - protein, 38 aa, Artificial Sequence
  Description of Artificial Sequence Exendin Agonist
HGEGTFTSDLSKQMEEEAVRLFIEWLKNGGPSSGAXXX

SEQ ID NO: 83 - protein, 37 aa, Artificial Sequence
  Description of Artificial Sequence Exendin Agonist
HGEGTFTSDLSKQMEEEAVRLFIEWLKNGGXSSGAPP

SEQ ID NO: 84 - protein, 37 aa, Artificial Sequence
  Description of Artificial Sequence Exendin Agonist
HGEGTFTSDLSKQMEEEAVRLFIEWLKNGGXSSGAXX

SEQ ID NO: 85 - protein, 37 aa, Artificial Sequence; identical to SEQ ID NO: 84
  Description of Artificial Sequence Exendin Agonist

SEQ ID NO: 86 - protein, 36 aa, Artificial Sequence
  Description of Artificial Sequence Exendin Agonist
HGEGTFTSDLSKQMEEEAVRLFIEWLKNGGXSSGAX

SEQ ID NO: 87 - protein, 35 aa, Artificial Sequence
  Description of Artificial Sequence Exendin Agonist
RGEGTFTSDLSKQMEEEAVRLFIEWLKNGGPSSGA

SEQ ID NO: 88 - protein, 30 aa, Artificial Sequence
  Description of Artificial Sequence Exendin Agonist
HGDGTFTSDLSKQMEEEAVRLFIEWLKNGG

SEQ ID NO: 89 - protein, 28 aa, Artificial Sequence
  Description of Artificial Sequence Exendin Agonist
HGEGTXTSDLSKQLEEEAVRLFIEFLKN

SEQ ID NO: 90 - protein, 28 aa, Artificial Sequence
  Description of Artificial Sequence Exendin Agonist
HGEGTFSSDLSKQMEEEAVRLFIEWLKN

SEQ ID NO: 91 - protein, 28 aa, Artificial Sequence
  Description of Artificial Sequence Exendin Agonist
HGEGTFSTDLSKQMEEEAVRLFIEWLKN

SEQ ID NO: 92 - protein, 28 aa, Artificial Sequence
  Description of Artificial Sequence Exendin Agonist
HGEGTFTSELSKQMAEEAVRLFIEWLKN

SEQ ID NO: 93 - protein, 28 aa, Artificial Sequence
  Description of Artificial Sequence Exendin Agonist
HGEGTFTSDXSKQLEEEAVRLFIEFLKN

SEQ ID NO: 94 - protein, 28 aa, Artificial Sequence
  Description of Artificial Sequence Exendin Agonist
HGEGTFTSDLSKQLEEEAVRLXIEFLKN

SEQ ID NO: 95 - protein, 28 aa, Artificial Sequence
  Description of Artificial Sequence Exendin Agonist
HGEGTFTSDLSKQMEEEAVRLFXEWLKN

SEQ ID NO: 96 - protein, 28 aa, Artificial Sequence
  Description of Artificial Sequence Exendin Agonist
HGEGTFTSDLSKQLEEEAVRLFIDFLKN

SEQ ID NO: 97 - protein, 33 aa, Artificial Sequence
  Description of Artificial Sequence Exendin Agonist
HGEGTFTSDASKQLEEEAVRLFIEFLKNGGPSS

SEQ ID NO: 98 - protein, 29 aa, Artificial Sequence
  Description of Artificial Sequence Exendin Agonist
HGEGTFTSDASKQMEEEAVRLFIEWLKNG

SEQ ID NO: 99 - protein, 37 aa, Artificial Sequence
  Description of Artificial Sequence Exendin Agonist
HGEGTFTSDASKQMEEEAVRLFIEWLKNGGXSSGAXX

SEQ ID NO: 100 - protein, 28 aa, Artificial Sequence
  Description of Artificial Sequence Exendin Agonist
AGEGTFTSDLSKQLEEEAVRLFIEFLKN

SEQ ID NO: 101 - protein, 28 aa, Artificial Sequence
  Description of Artificial Sequence Exendin Agonist
HGAGTFTSDLSKQLEEEAVRLFIEFLKN

SEQ ID NO: 102 - protein, 28 aa, Artificial Sequence
  Description of Artificial Sequence Exendin Agonist
HGEATFTSDLSKQLEEEAVRLFIEFLKN

SEQ ID NO: 103 - protein, 28 aa, Artificial Sequence
  Description of Artificial Sequence Exendin Agonist
HGEGTFTSALSKQLEEEAVRLFIEFLKN

SEQ ID NO: 104 - protein, 28 aa, Artificial Sequence
  Description of Artificial Sequence Exendin Agonist
AGEGTFTSDLSKQMEEEAVRLFIEWLKN

SEQ ID NO: 105 - protein, 28 aa, Artificial Sequence
  Description of Artificial Sequence Exendin Agonist
HGAGTFTSDLSKQMEEEAVRLFIEWLKN

SEQ ID NO: 106 - protein, 28 aa, Artificial Sequence
  Description of Artificial Sequence Exendin Agonist
HGEATFTSDLSKQMEEEAVRLFIEWLKN

SEQ ID NO: 107 - protein, 28 aa, Artificial Sequence
  Description of Artificial Sequence Exendin Agonist
HGEGTFTSALSKQMEEEAVRLFIEWLKN

SEQ ID NO: 108 - protein, 28 aa, Artificial Sequence
  Description of Artificial Sequence Exendin Agonist
HGEGTFTSDASKQMEEEAVRLFIEWLKN

SEQ ID NO: 109 - protein, 28 aa, Artificial Sequence
  Description of Artificial Sequence Exendin Agonist
AAEGTFTSDLSKQMEEEAVRLFIEWLKN

SEQ ID NO: 110 - protein, 28 aa, Artificial Sequence
  Description of Artificial Sequence Exendin Agonist
AAEGTFTSDLSKQLEEEAVRLFIEFLKN

SEQ ID NO: 111 - protein, 28 aa, Artificial Sequence
  Description of Artificial Sequence Exendin Agonist
AGDGTFTSDLSKQMEEEAVRLFIEWLKN

SEQ ID NO: 112 - protein, 28 aa, Artificial Sequence
  Description of Artificial Sequence Exendin Agonist
AGDGTFTSDLSKQLEEEAVRLFIEFLKN

SEQ ID NO: 113 - protein, 28 aa, Artificial Sequence
  Description of Artificial Sequence Exendin Agonist
AGDGAFTSDLSKQMEEEAVRLFIEWLKN

SEQ ID NO: 114 - protein, 28 aa, Artificial Sequence
  Description of Artificial Sequence Exendin Agonist
AGDGAFTSDLSKQLEEEAVRLFIEFLKN

SEQ ID NO: 115 - protein, 28 aa, Artificial Sequence
  Description of Artificial Sequence Exendin Agonist
AGDGTXTSDLSKQMEEEAVRLFIEWLKN

SEQ ID NO: 116 - protein, 28 aa, Artificial Sequence
  Description of Artificial Sequence Exendin Agonist
AGDGTXTSDLSKQLEEEAVRLFIEFLKN

SEQ ID NO: 117 - protein, 28 aa, Artificial Sequence
  Description of Artificial Sequence Exendin Agonist
AGDGTFSSDLSKQMEEEAVRLFIEWLKN

SEQ ID NO: 118 - protein, 28 aa, Artificial Sequence
  Description of Artificial Sequence Exendin Agonist
AGDGTFSSDLSKQLEEEAVRLFIEFLKN

SEQ ID NO: 119 - protein, 28 aa, Artificial Sequence
  Description of Artificial Sequence Exendin Agonist
AGDGTFTADLSKQMEEEAVRLFIEWLKN

SEQ ID NO: 120 - protein, 28 aa, Artificial Sequence
  Description of Artificial Sequence Exendin Agonist
AGDGTFTADLSKQLEEEAVRLFIEFLKN

SEQ ID NO: 121 - protein, 28 aa, Artificial Sequence
  Description of Artificial Sequence Exendin Agonist
AGDGTFTSALSKQMEEEAVRLFIEWLKN

SEQ ID NO: 122 - protein, 28 aa, Artificial Sequence
  Description of Artificial Sequence Exendin Agonist
AGDGTFTSALSKQLEEEAVRLFIEFLKN

SEQ ID NO: 123 - protein, 28 aa, Artificial Sequence
  Description of Artificial Sequence Exendin Agonist
AGDGTFTSELSKQMEEEAVRLFIEWLKN

SEQ ID NO: 124 - protein, 28 aa, Artificial Sequence
  Description of Artificial Sequence Exendin Agonist
AGDGTFTSELSKQLEEEAVRLFIEFLKN

SEQ ID NO: 125 - protein, 28 aa, Artificial Sequence
  Description of Artificial Sequence Exendin Agonist
AGDGTFTSDASKQMEEEAVRLFIEWLKN

SEQ ID NO: 126 - protein, 28 aa, Artificial Sequence
  Description of Artificial Sequence Exendin Agonist
AGDGTFTSDASKQLEEEAVRLFIEFLKN

SEQ ID NO: 127 - protein, 28 aa, Artificial Sequence
  Description of Artificial Sequence Exendin Agonist
AGDGTFTSDXSKQMEEEAVRLFIEWLKN

SEQ ID NO: 128 - protein, 28 aa, Artificial Sequence
  Description of Artificial Sequence Exendin Agonist
AGDGTFTSDXSKQLEEEAVRLFIEFLKN

SEQ ID NO: 129 - protein, 28 aa, Artificial Sequence
  Description of Artificial Sequence Exendin Agonist
AGDGTFTSDLAKQMEEEAVRLFIEWLKN

SEQ ID NO: 130 - protein, 28 aa, Artificial Sequence
  Description of Artificial Sequence Exendin Agonist
AGDGTFTSDLAKQLEEEAVRLFIEFLKN

SEQ ID NO: 131 - protein, 28 aa, Artificial Sequence
  Description of Artificial Sequence Exendin Agonist
AGDGTFTSDLSAQMEEEAVRLFIEWLKN

SEQ ID NO: 132 - protein, 28 aa, Artificial Sequence
  Description of Artificial Sequence Exendin Agonist
AGDGTFTSDLSAQLEEEAVRLFIEFLKN

SEQ ID NO: 133 - protein, 28 aa, Artificial Sequence
  Description of Artificial Sequence Exendin Agonist
AGDGTFTSDLSKAMEEEAVRLFIEWLKN

SEQ ID NO: 134 - protein, 28 aa, Artificial Sequence
  Description of Artificial Sequence Exendin Agonist
AGDGTFTSDLSKALEEEAVRLFIEFLKN

SEQ ID NO: 135 - protein, 28 aa, Artificial Sequence
  Description of Artificial Sequence Exendin Agonist
AGDGTFTSDLSKQAEEEAVRLFIEWLKN

SEQ ID NO: 136 - protein, 28 aa, Artificial Sequence
  Description of Artificial Sequence Exendin Agonist
AGDGTFTSDLSKQAEEEAVRLFIEFLKN

SEQ ID NO: 137 - protein, 28 aa, Artificial Sequence
  Description of Artificial Sequence Exendin Agonist
AGDGTFTSDLSKQXEEEAVRLFIEWLKN

SEQ ID NO: 138 - protein, 28 aa, Artificial Sequence
  Description of Artificial Sequence Exendin Agonist
AGDGTFTSDLSKQXEEEAVRLFIEFLKN

SEQ ID NO: 139 - protein, 28 aa, Artificial Sequence
  Description of Artificial Sequence Exendin Agonist
AGDGTFTSDLSKQMAEEAVRLFIEWLKN

SEQ ID NO: 140 - protein, 28 aa, Artificial Sequence
  Description of Artificial Sequence Exendin Agonist
AGDGTFTSDLSKQLAEEAVRLFIEFLKN

SEQ ID NO: 141 - protein, 28 aa, Artificial Sequence
  Description of Artificial Sequence Exendin Agonist
AGDGTFTSDLSKQMEAEAVRLFIEWLKN

SEQ ID NO: 142 - protein, 28 aa, Artificial Sequence
  Description of Artificial Sequence Exendin Agonist
AGDGTFTSDLSKQLEAEAVRLFIEFLKN

SEQ ID NO: 143 - protein, 28 aa, Artificial Sequence
  Description of Artificial Sequence Exendin Agonist
AGDGTFTSDLSKQMEEAAVRLFIEWLKN

SEQ ID NO: 144 - protein, 28 aa, Artificial Sequence
  Description of Artificial Sequence Exendin Agonist
AGDGTFTSDLSKQLEEAAVRLFIEFLKN

SEQ ID NO: 145 - protein, 28 aa, Artificial Sequence
  Description of Artificial Sequence Exendin Agonist
AGDGTFTSDLSKQMEEEAARLFIEWLKN

SEQ ID NO: 146 - protein, 28 aa, Artificial Sequence
  Description of Artificial Sequence Exendin Agonist
AGDGTFTSDLSKQLEEEAARLFIEFLKN

SEQ ID NO: 147 - protein, 28 aa, Artificial Sequence
  Description of Artificial Sequence Exendin Agonist
AGDGTFTSDLSKQMEEEAVALFIEWLKN

SEQ ID NO: 148 - protein, 28 aa, Artificial Sequence
  Description of Artificial Sequence Exendin Agonist
AGDGTFTSDLSKQLEEEAVALFIEFLKN

SEQ ID NO: 149 - protein, 28 aa, Artificial Sequence
  Description of Artificial Sequence Exendin Agonist
AGDGTFTSDLSKQMEEEAVRAFIEWLKN

SEQ ID NO: 150 - protein, 28 aa, Artificial Sequence
  Description of Artificial Sequence Exendin Agonist
AGDGTFTSDLSKQLEEEAVRAFIEFLKN

SEQ ID NO: 151 - protein, 28 aa, Artificial Sequence
  Description of Artificial Sequence Exendin Agonist
AGDGTFTSDLSKQMEEEAVRLXIEWLKN

SEQ ID NO: 152 - protein, 28 aa, Artificial Sequence
  Description of Artificial Sequence Exendin Agonist
AGDGTFTSDLSKQLEEEAVRLXIEFLKN

SEQ ID NO: 153 - protein, 28 aa, Artificial Sequence
  Description of Artificial Sequence Exendin Agonist
AGDGTFTSDLSKQMEEEAVRLFVEWLKN

SEQ ID NO: 154 - protein, 28 aa, Artificial Sequence
  Description of Artificial Sequence Exendin Agonist
AGDGTFTSDLSKQLEEEAVRLFVEFLKN

SEQ ID NO: 155 - protein, 28 aa, Artificial Sequence
  Description of Artificial Sequence Exendin Agonist
AGDGTFTSDLSKQMEEEAVRLFXEWLKN

SEQ ID NO: 156 - protein, 28 aa, Artificial Sequence
  Description of Artificial Sequence Exendin Agonist
AGDGTFTSDLSKQLEEEAVRLFXEFLKN

SEQ ID NO: 157 - protein, 28 aa, Artificial Sequence
  Description of Artificial Sequence Exendin Agonist
AGDGTFTSDLSKQMEEEAVRLFIDWLKN

SEQ ID NO: 158 - protein, 28 aa, Artificial Sequence
  Description of Artificial Sequence Exendin Agonist
AGDGTFTSDLSKQLEEEAVRLFIDFLKN

SEQ ID NO: 159 - protein, 28 aa, Artificial Sequence
  Description of Artificial Sequence Exendin Agonist
AGDGTFTSDLSKQMEEEAVRLFIEALKN

SEQ ID NO: 160 - protein, 28 aa, Artificial Sequence
  Description of Artificial Sequence Exendin Agonist
AGDGTFTSDLSKQLEEEAVRLFIEALKN

SEQ ID NO: 161 - protein, 28 aa, Artificial Sequence
  Description of Artificial Sequence Exendin Agonist
AGDGTFTSDLSKQMEEEAVRLFIEWAKN

SEQ ID NO: 162 - protein, 28 aa, Artificial Sequence
  Description of Artificial Sequence Exendin Agonist
AGDGTFTSDLSKQLEEEAVRLFIEFAKN

SEQ ID NO: 163 - protein, 28 aa, Artificial Sequence
  Description of Artificial Sequence Exendin Agonist
AGDGTFTSDLSKQMEEEAVRLFIEWLAN

SEQ ID NO: 164 - protein, 28 aa, Artificial Sequence
  Description of Artificial Sequence Exendin Agonist
AGDGTFTSDLSKQLEEEAVRLFIEFLAN

SEQ ID NO: 165 - protein, 28 aa, Artificial Sequence
  Description of Artificial Sequence Exendin Agonist
AGDGTFTSDLSKQMEEEAVRLFIEWLKA

SEQ ID NO: 166 - protein, 28 aa, Artificial Sequence
  Description of Artificial Sequence Exendin Agonist
AGDGTFTSDLSKQLEEEAVRLFIEFLKA

SEQ ID NO: 167 - protein, 38 aa, Artificial Sequence
  Description of Artificial Sequence Exendin Agonist
AGEGTFTSDLSKQMEEEAVRLFIEWLKNGGPSSGAPPP

SEQ ID NO: 168 - protein, 38 aa, Artificial Sequence
  Description of Artificial Sequence Exendin Agonist
HGAGTFTSDLSKQLEEEAVRLFIEFLKNGGPSSGAPPP

SEQ ID NO: 169 - protein, 37 aa, Artificial Sequence
  Description of Artificial Sequence Exendin Agonist
HGEATFTSDLSKQMEEEAVRLFIEWLKNGGPSSGAPP

SEQ ID NO: 170 - protein, 36 aa, Artificial Sequence
  Description of Artificial Sequence Exendin Agonist
HGEGTFTSALSKQMEEEAVRLFIEWLKNGGPSSGAP

SEQ ID NO: 171 - protein, 36 aa, Artificial Sequence
  Description of Artificial Sequence Exendin Agonist
AGEGTFTSDASKQLEEEAVRLFIEFLKNGGPSSGAP

SEQ ID NO: 172 - protein, 35 aa, Artificial Sequence
  Description of Artificial Sequence Exendin Agonist
AGEGTFTSDLSKQMEEEAVRLFIEWLKNGGPSSGA

SEQ ID NO: 173 - protein, 35 aa, Artificial Sequence
  Description of Artificial Sequence Exendin Agonist
HGAGTFTSDLSKQLEEEAVRLFIEFLKNGGPSSGA

SEQ ID NO: 174 - protein, 34 aa, Artificial Sequence
  Description of Artificial Sequence Exendin Agonist
HGEATFTSDLSKQMEEEAVRLFIEWLKNGGPSSG

SEQ ID NO: 175 - protein, 33 aa, Artificial Sequence
  Description of Artificial Sequence Exendin Agonist
HGEGTFTSALSKQMEEEAVRLFIEWLKNGGPSS

SEQ ID NO: 176 - protein, 32 aa, Artificial Sequence
  Description of Artificial Sequence Exendin Agonist
AGEGTFTSDLSKQMEEEAVRLFIEWLKNGGPS

SEQ ID NO: 177 - protein, 32 aa, Artificial Sequence
  Description of Artificial Sequence Exendin Agonist
HGAGTFTSDLSKQLEEEAVRLFIEFLKNGGPS

SEQ ID NO: 178 - protein, 31 aa, Artificial Sequence
  Description of Artificial Sequence Exendin Agonist
HGEATFTSDLSKQMEEEAVRLFIEWLKNGGP

SEQ ID NO: 179 - protein, 30 aa, Artificial Sequence
  Description of Artificial Sequence Exendin Agonist
HGEGTFTSALSKQLEEEAVRLFIEFLKNGG

SEQ ID NO: 180 - protein, 29 aa, Artificial Sequence
  Description of Artificial Sequence Exendin Agonist
AGEGTFTSDLSKQLEEEAVRLFIEFLKNG

SEQ ID NO: 181 - protein, 38 aa, Artificial Sequence
  Description of Artificial Sequence Exendin Agonist
HGAGTFTSDLSKQMEEEAVRLFIEWLKNGGXSSGAXXX

SEQ ID NO: 182 - protein, 38 aa, Artificial Sequence
  Description of Artificial Sequence Exendin Agonist
HGEATFTSDLSKQMEEEAVRLFIEWLKNGGPSSGAPPP

SEQ ID NO: 183 - protein, 37 aa, Artificial Sequence
  Description of Artificial Sequence Exendin Agonist
HGEGTFTSALSKQMEEEAVRLFIEWLKNGGXSSGAXX

SEQ ID NO: 184 - protein, 36 aa, Artificial Sequence
  Description of Artificial Sequence Exendin Agonist
AGEGTFTSDLSKQMEEEAVRLFIEWLKNGGXSSGAX

SEQ ID NO: 185 - protein, 35 aa, Artificial Sequence
  Description of Artificial Sequence Exendin Agonist
HGAGTFTSDLSKQMEEEAVRLFIEWLKNGGPSSGA

SEQ ID NO: 186 - protein, 30 aa, Artificial Sequence
  Description of Artificial Sequence Exendin Agonist
HGDATFTSDLSKQMEEEAVRLFIEWLKNGG

SEQ ID NO: 187 - protein, 39 aa, Artificial Sequence
  Description of Artificial Sequence Exendin Agonist
AGEGTFTSDLSKQMEEEAVRLFIEWLKNGGPSSGAPPPS

SEQ ID NO: 188 - protein, 39 aa, Artificial Sequence
  Description of Artificial Sequence Exendin Agonist
AGAGTFTSDLSKQLEEEAVRLFIEFLKNGGPSSGAPPPS